(19) United States
(12) Patent Application Publication
(10) Pub. No.: US 2010/0040608 A1
(43) Pub. Date: Feb. 18, 2010

(54) Use of HMGB1 antagonists for the treatment of inflammatory skin conditions

(76) Inventors: Marie Wahren-Herlenius, Djursholm (SE); Filippa Nyberg, Stocksund (SE); Giovanna Marchini, Stockholm (SE)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/988,896

(22) PCT Filed: Jul. 11, 2006
(86) PCT No.: PCT/US2006/027053

§ 371 Date: May 5, 2009

Related U.S. Application Data
(60) Provisional application No. 60/700,704, filed on Jul. 18, 2005.

(51) Int. Cl. A61K 39/395; A61K 38/17

(52) U.S. Cl. 424/133.1; 424/130.1; 424/139.1; 514/12

(57) ABSTRACT

Methods are disclosed for treating an inflammatory skin condition in a subject. The methods comprise administering to a subject an HMGB antagonist, such as a high mobility group box (HMGB) A box or a biologically active fragment thereof, an antibody to HMGB or an antigen-binding fragment thereof, an HMGB small molecule antagonist, an antibody to TLR2 or an antigen-binding fragment thereof, a soluble TLR2 polypeptide, an antibody to RAGE or an antigen-binding fragment thereof, a soluble RAGE polypeptide and a RAGE small molecule antagonist.

### RELATED APPLICATIONS

This application claims the benefit of U.S. Provisional Application No. 60/700,704, filed on Jul. 18, 2005. The entire teachings of the above application are incorporated herein by reference.

### BACKGROUND OF THE INVENTION

Inflammation is often induced by proinflammatory cytokines, such as tumor necrosis factor (TNF), interleukin (IL)-1α, IL-1β, IL-6, macrophage migration inhibitory factor (MIF), and other compounds. These proinflammatory cytokines are produced by several different cell types, including immune cells (for example, monocytes, macrophages and neutrophils) and non-immune cells, such as fibroblasts, osteoblasts, smooth muscle cells, epithelial cells and neurons. These proinflammatory cytokines contribute to various disorders during the early stages of an inflammatory cytokine cascade.

During autoimmune inflammation, pro- and anti-inflammatory cytokines are produced (Dinarello, C. A., Chest 118:503-08 (2000)). TNF-α and IL-1β are pro-inflammatory cytokines that have been shown to be of central importance in several autoimnmune conditions, including rheumatoid arthritis, myositis and Sjögren's syndrome. TNF-α is predominately synthesized by macrophages/monocytes (Dinarello, C. A., J. Exp. Med. 163:1433-50 (1986)), although keratinocytes also exhibit the capacity to release TNF-α (Köck A. et al., J. Exp. Med. 172:1609-14 (1990)). Another major source of TNF-α in the skin are mast cells. For example, ultraviolet B light (UVB) can induce mast cells to degranulate and release their intracellular stores of TNF-α (Walsh et al., Immunol. Cell Biol. 73:226-233 (1995)). Ultraviolet radiation (UV R) causes the release of both IL-1 and TNF-α from the epidermis (Dinarello, C. A., Chest 118:503-08 (2000)) and photosensitivity has been demonstrated in many studies.

The high mobility group box chromosomal protein 1 (HMGB1) is an intranuclear protein, which binds DNA and is involved in the organization of chromatin (Bustin M., Mol. Cell. Biol. 19:5237-46 (1999)). More recently, HMGB1 was found to act as a pro-inflammatory cytokine (Yang H., et al., Shock 15:247-53 (2001)), and to be actively secreted by macrophages/monocytes by inflammatory stimuli (Wang H., et al., Science 285:248-51 (1999)). During secretion, HMGB1 exits the nucleus and is transported through the cytoplasm, where it is actively released to the extracellular space. HMGB1 can also be passively released from the nuclei of necrotic or damaged cells (Scaffidi P., et al., Nature 418:191-95 (2002)). Both TNF-α and IL-1β have been shown to stimulate the release of HMGB1 (Wang H., et al., Surgery 126:389-92 (1999)), and HMGB1 may in turn stimulate the synthesis of pro-inflammatory cytokines (Andersson, U., et al., J. Exp. Med. 192:565-570 (2000)).

Inflammatory skin disorders affect millions of people annually in the United States alone. On a worldwide scale this figure is staggering. Such disorders range from the relatively minor inconvenience of dry skin to more serious life-threatening conditions. For many inflammatory skin conditions (e.g., acne, pruritis, rosacea, erythematosus multiforme, erythema toxicum, folliculitis, impetigo, cutaneous lupus erythematosus (CLE), cold sores, dry skin and insect bites), there are insufficient or inadequate treatments.

One such inflammatory skin condition is acne. Acne is the most common pustular condition of the skin, and can result in inflammatory and noninflammatory lesions (including pustules, papules and comedones) during its active phase, with atrophic scars afterwards. It occurs most commonly in teenagers, but is not confined to adolescents, as increasing numbers of people older than 20 are seeking advice for treatment for acne (Brogden, R. N., and Goa, K. L., Drugs 53:511-519 (1997)). Although acne is generally considered to be self-limiting, its social effects can be substantial, and it may have severe psychological effects.

Acne is a multifactorial disease affecting the pilosebaceous units of the skin. Each unit consists of a large, multilobed sebaceous gland, a rudimentary hair and a wide follicular canal lined with stratefied squamous epithelium. They are found over most of the body surface but are largest and most numerous on the face, chest, and upper back. Normally, desquamated follicular cells are carried to the surface by the flow of sebum. Under the abnormal circumstances of acne vulgaris, an abnormal desquamination process provokes increased sloughing of the epithelium, which becomes more cohesive because of defective keratinization. This process causes blockage of the follicular orifice with accumulation of dead cells. Androgen stimulates the undifferentiated hormonally responsive cells making up the outer layer of the sebaceous gland lobule to divide and differentiate. Sebum production favors proliferation of the anaerobe Propionibacterium acnes, which is a normal commensal to the pilosebaceous unit, but can elicit hypersensitivity responses in acne.

The basic lesion of acne is the microcomedo. Accumulation of sebum and keratinous debris results in a visible closed comedo, or whitehead, and its continued distension causes an open comedo, or blackhead. The dark color of blackheads is due to oxidized melanin. Blackheads and microcysts are noninflammatory lesions of acne, but some comedones evolve into inflammatory papules, pustules, or nodules, and can become chronic granulomatous lesions. The initial inflammatory cell in an acute acne papule is the CD4⁺T lymphocyte. Duct rupture is not a prerequisite for inflammation, which is due to the release of pro-inflammatory substances from the duct. When inflammation develops, neutrophil chemotaxis occurs. These neutrophils secrete hydrolytic enzymes that cause further damage and increased permeability of the follicular wall. In pustules, neutrophils are present much earlier. More persistent lesions exhibit granulomatous histology that can lead to scarring.

There are several known antibiotic substances that are effective against Propionibacteria and also have anti-inflammatory properties. However, in pretreated, as well as in non-pretreated acne patients, a drastic increase has been observed in the overall resistance of the Propionibacteria to antibiotics. In certain circumstances, a resistance rate of up to 60% to one or more antibiotics has been observed.

Another inflammatory skin condition is rosacea. Rosacea, originally termed acne rosacea, is a chronic inflammatory skin condition affecting the eyelids and face, particularly the cheeks, chin, nose, and forehead. Common clinical signs include erythema (redness), prominent vascularization, dryness, papules, pustules, swelling, telangiectasia, lesions, inflammation, infection, enlarged nasal area, hypertrophy of the sebaceous glands, and nodules either singly or in combination in the involved skin areas, primarily in the central areas of the face. Some of these clinical signs, in particular the erythema, are thought to be caused by the dilation of blood vessels. Rosacea may further be characterized by flushing and blushing. In rare instances, rosacea may also occur on the trunk and extremities, such as the chest, neck, back, or scalp.

Rosacea, in mild form, brings about a slight flushing of the nose and cheeks and, in some cases, the forehead and chin. However, in a severe form, lesions appear which are deep or purplish red and which include a chronic dilation of the superficial capillaries. Also, in severe form, inflammatory acneiform pustules are present. Chronic involvement of the nose with rosacea in men can cause a bulbous enlargement known as rhinophyma. However, women are twice as likely as men to have rosacea. In women, this rhinophyma often takes the form of pimples and redness of, or near, the nose. Similarly, women are three times more likely than men to exhibit symptoms of perioral dermatitis, where redness and a rash appear above the upper lip.

Rosacea has also been treated with oral and/or topical antibacterial agents. Such oral antibiotics include tetracycline, erythromycin and minocycline. This antibiotic treatment has been shown to effectively block progression of rosacea through a poorly-understood anti-inflammatory mechanism, but studies have shown that these medications do not act by killing either bacteria or Demodex folliculorum organisms in affected skin.

Still another inflammatory skin condition is cutaneous lupus erythematosus (CLE). Subacute cutaneous lupus erythematosus (SCLE) and chronic cutaneous lupus erythematosus (CCLE) are two subsets of CLE. SCLE is defined as a non-scarring skin eruption in association with Ro/SSA-autoantibodies and photosensitivity (Sontheimer, R. D., Med. Clin. N. Am. 73(5):1073-1090 (1989)). The skin lesions of SCLE can be papulosquamous or annular and are most commonly distributed on the neck, shoulders and extensor surfaces of upper extremities. Histologically, the lesions show hydropic degeneration of the basal layer of the epidermis, and in the dermis a mononuclear infiltrate is seen.

The most common form of CCLE is discoid lupus erythematosus (DLE). The skin lesion of DLE typically present as red plaques with thick scale and follicular plugs. The lesions heal with atrophy, scarring and depigmentation. Histologically, the lesions show epidermal atrophy, hydropic degeneration of the basal layer of the epidermis, mononuclear peri-appendageal infiltrates and follicular plugging. The inflammatory cells in DLE lesions have been reported to be predominately CD3⁺ T cells with CD4⁺ helper T cells present in higher numbers than CD8⁺ cytotoxic T cells (Kuhn, A., et al., Arch. Dermatol. Res. 294(1-2):6-13 (2002)).

In addition to the conditions described above, other inflammatory skin conditions also require improved methods of treatment. Accordingly, there is a need for safe and effective agents that are useful in treating such inflammatory skin conditions.

### SUMMARY OF THE INVENTION

As described herein, the present invention is based on the discoveries that the pro-inflammatory cytokine, HMGB1, is secreted by keratinocytes; and that its expression increases in inflammatory skin conditions. Thus, in one embodiment, the invention is a method of treating an inflammatory skin condition in a subject by administering to the subject an HMGB antagonist.

In one embodiment, the invention is a method of treating an inflammatory skin condition selected from the group consisting of psoriasis, acne, pruritis, rosacea, dermatitis, erythematosus multiforme, erythema toxicum, folliculitis, impetigo, cutaneous lupus erythematosus (CLE), cold sores, dry skin, allergic skin conditions, burns, sunburn and insect bites by administering to a subject an HMGB antagonist. In another embodiment, the condition to be treated is dermatitis (e.g., atopic dermatitis, contact dermatitis, seborrheic dermatitis, nummular dermatitis, exfoliative dermatitis, periorial dermatitis, stasis dermatitis).

In one embodiment, the invention is a method of treating a bacterially-mediated inflammatory skin condition in a subject by administering an HMGB antagonist. In another embodiment, the bacterially-mediated inflammatory skin condition to be treated is selected from the group consisting of acne, rosacea, cellulitis, acute lymphangitis, lymphadenitis, erysipelas, cutaneous abcesses, necrotizing subcutaneous infections, staphylococcal scalded skin syndrome, folliculitis, furuncles, hidradenitis suppurativa, carbuncles, paronychial infections and erythasma, nummular dermatitis and perioral dermatitis. In still another embodiment, the bacterially-mediated inflammatory skin condition is acne or rosacea.

In one embodiment, the invention is a method of treating cutaneous lupus erthematosus (CLE) (e.g., acute cutaneous lupus erthematosus (ACLE), subacute (CCLE) (e.g., discoid lupus erthematosus (DLE))) in a subject by administering an HMGB1 antagonist.

In another embodiment, the invention is a method of treating erythema toxicum in a subject by administering an HMGB1 antagonist.

In another embodiment, the invention is a method of inhibiting release of HMGB1 from keratinocytes comprising administering an HMGB1 antagonist.

In another embodiment, the invention is a method of treating melanoma in a subject by administering to the subject an HMGB1 antagonist.

In still another embodiment, the invention is a method of treating lupus erthematosus (LE) (e.g., cutaneous lupus erthematosus (CLE), systemic lupus erthematosus, drug-induced lupus erthematosus, neonatal lupus erythematosus) in a subject by administering an HMGB1 antagonist.

In yet another embodiment, the invention is a method of preventing or decreasing tissue damage (e.g., skin damage) from exposure to ultraviolet radiation (UV R) by administering an HMGB antagonist.

In particular embodiments, the HMGB antagonist used in the methods of the invention is selected from the group consisting of a high mobility group box (HMGB) A box or a biologically active fragment thereof, an antibody to HMGB or an antigen-binding fragment thereof, an HMGB small molecule antagonist, an antibody to TLR2 or an antigen-binding fragment thereof, a soluble TLR2-polypeptide, an antibody to RAGE or an antigen-binding fragment thereof, a soluble RAGE polypeptide and a RAGE small molecule antagonist.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A is a section of a skin biopsy from a patient with an SCLE lesion. The section has been stained with HMGB1 antibodies. The image magnification is 25×.

FIG. 1B is a section from the unaffected buttock skin of the same patient as FIG. 1A. The section has been stained with HMGB1 antibodies. The image magnification is 25×.

FIG. 1C is a section from the buttock skin of a healthy control patient. The section has been stained with HMGB1 antibodies. The image magnification is 25×.

FIG. 1D is a box-plot representation showing a semi-quantitative analysis of HMGB1 expression in the epidermis (n=10, p<0.01) and dermis (n=10, p<0.001) of affected and unaffected skin in the same patients (n=10, p<0.01), respectively.

FIG. 2A is a section of a lesion from a patient with SCLE. The section has been stained with HMGB1 antibodies. Extracellular staining of HMGB1 is indicated by a black arrow. The image magnification is 80×.

FIG. 2B is a section from the unaffected buttock skin of the same patient as FIG. 2A. The section has been stained with HMGB1 antibodies. Cytoplasmic staining is indicated by a black arrow and nuclear staining is indicated by a white arrow. The image magnification is 80×.

FIG. 2C is a section of buttock skin from a healthy control individual. The section has been stained with HMGB1 antibodies. Cytoplasmic staining is indicated by a black arrow and nuclear staining is indicated by a white arrow. The image magnification is 80×.

FIG. 2D is a box-plot representation showing a semi-quantitative analysis of HMGB1 expression in the epidermis (n=10, p<0.01) and dermis (n=10, p<0.001) of affected and unaffected skin in the same patients (n=10, p<0.01), respectively.

FIG. 3A shows expression of TNF-α in a section of a skin biopsy from an SCLE lesion. The image magnification is 25×.

FIG. 3B shows expression of TNF-α in a section of a skin biopsy from the healthy buttock skin of the same patient as FIG. 3A. The image magnification is 25×.

FIG. 3C shows expression of IL-1β in a section of a skin biopsy from an SCLE lesion. The image magnification is 25×.

FIG. 3D shows expression of IL-1β in a section of a skin biopsy from the healthy buttock skin of the sane patient as FIG. 3C. The image magnification is 25×.

FIG. 4 is a box-plot representation depicting the percentage of HMGB1 positive cells in the epidermis of CLE patients before and after UVB exposure. Four time points are presented: 1=before UVB exposure, 2=flare after UVB exposure, 3=follow up, 4=disolvance of lesion. Statistically significant differences are indicated by a star (*) symbol.

FIG. 5 is a box-plot representation depicting the percentage of HMGB1 staining in the cytoplasm of epidermis cells of CLE patients before and after UVB exposure. Four time points are presented: 1=before UVB exposure, 2=flare after UVB exposure, 3=follow up, 4=disolvance of lesion. Statistically significant differences are indicated by a star (*) symbol.

FIG. 6 is a box-plot representation depicting changes in the percentage of cytoplasmic or nuclear HMGB1 staining in keratinocytes from the healthy skin or UVB-induced lesion flares of CLE patients. Only changes in cytoplasmic staining were significant (p<0.05).

FIG. 7 is a box-plot representation depicting changes in the expression of HMGB1 in the extracellular space of the epidermis in the healthy skin Or UVB-induced lesion flares of CLE patients.

FIG. 8 is a box-plot representation depicting the percentage of staining in the dermis cells of CLE patients before and after UVB exposure. Four time points are presented: 1=before UVB exposure, 2=flare after UVB exposure, 3=follow up, 4=disolvance of lesion. Statistically significant differences are indicated by a star (*) symbol.

FIG. 9 is a box-plot representation depicting the percentage of HMGB1 staining in the cytoplasm in non-infiltrated dermis cells of CLE patients before and after UVB exposure. Four time points are presented: 1=before UVB exposure, 2=flare after UVB exposure, 3-follow up, 4=disolvance of lesion. Statistically. significant differences are indicated by a star (*) symbol.

FIG. 10 is a box-plot representation depicting the percentage of nuclear HMGB1 staining in non-infiltrated dermis cells of CLE patients before and after UVB exposure. Four time points are presented: 1=before UVB exposure, 2=flare after UVB exposure, 3=follow up, 4=disolvance of lesion. Statistically significant differences are indicated by a star (*) symbol.

FIG. 11 is a box-plot representation depicting changes in the percentage of cytoplasmic or nuclear HMGB1 staining in the dermis of healthy skin or UVB-induced lesion flares of CLE patients. Only changes in cytoplasmic staining were significant (p<0.05).

FIG. 12 is a photograph of the back of a 1-day-old infant with a typical generalized Erythema Toxicum rash.

FIG. 13A shows a section of an Erythema Toxicum lesion that has been stained with HMGB1 antibodies.

FIG. 13B is an enlarged view of the boxed-region on the left side of FIG. 13A, showing staining in the cytoplasm of, and extracellular space surrounding, keratinocytes that are located near the opening of a hair follicle.

FIG. 13C is an enlarged view of the boxed-region on the right side of FIG. 13A, showing HMGB1 staining in perifollicular inflammatory cells from a section of an Erythema Toxicum lesion.

FIG. 13D shows kerafinocytes overriding a hair follicle in a section from a lesion of Erythema Toxicum that has been stained with HMGB1 antibodies. Note the passage from cytoplasmic staining of HMGB1 on the right side of the section, to nuclear staining on the left side of the section.

FIG. 13E shows HMGB1 staining in keratinocytes surrounding a non-inflamed hair follicle from a section of a skin biopsy from a healthy control infant.

FIG. 14A shows a confocal micrograph of a section of the epidermal layer of a lesion of Erythema Toxicum that has been stained with DAPI.

FIG. 14B shows a confocal micrograph of a section of the epidermal layer of a lesion of Erythema Toxicum that has been stained with HMGB1 antibodies.

FIG. 14C shows a merged image of FIGS. 14A and 14B.

FIG. 14D is an enlarged view of the boxed region in FIG. 14C, showing DAPI staining.

FIG. 14E is an enlarged view of the boxed region in FIG. 14C, showing staining with HMGB1 antibodies.

FIG. 14F is an enlarged view of the boxed region in FIG. 14C, showing a merged image of FIGS. 14A and 14B.

FIG. 14G shows a confocal micrograph of a section of the epidermal layer from non-inflamed skin that has been stained with DAPI.

FIG. 14H shows a confocal micrograph of a section of the epidermal layer from non-inflamed skin that has been stained with HMGB1 antibodies.

FIG. 14I shows a merged image of FIGS. 14G and 14H.

FIG. 14J is an enlarged view of the boxed region in FIG. 14I, showing DAPI staining.

FIG. 14K is an enlarged view of the boxed region in FIG. 14L, showing staining with HMGB1 antibodies.

FIG. 14L is an enlarged view of the boxed region in FIG. 14I, showing a merged image of FIGS. 14J and 14K.

FIG. 15A is a confocal micrograph showing DAPI counterstaining of a MAC387-expressing macrophage from the perifollicular infiltrate of an Erythema Toxicum lesion.

FIG. 15B is a confocal micrograph showing HMGB1 immunostaining of a MAC387-expressing macrophage, from the perifollicular infiltrate of an Erythema Toxicum lesion.

FIG. 15C is a confocal micrograph showing MAC387 staining of a MAC387-expressing macrophage from the perifollicular infiltrate of an Erythema Toxicum lesion.

FIG. 15D is a merged image of FIG. 15A-15C.

FIG. 15E is a confocal micrograph showing DAPI counterstaining of a MAC387-expressing macrophage from the same biopsy section as FIG. 15A-15D.

FIG. 15F is a confocal micrograph showing HMGB1 immunostaining of a MAC387-expressing macrophage from the same biopsy section as FIG. 15A-15D.

FIG. 15G is a confocal micrograph showing MAC387 staining of a MAC387-expressing macrophage from the same biopsy section as FIG. 15A-15D.

FIG. 15H is a merged image of FIG. 15E-15G.

FIG. 16A shows a confocal micrograph of a section of the epidermal layer in a lesion of Erythema Toxicum that has been counterstained with DAPI.

FIG. 16B shows a confocal micrograph of a section of the epidermal layer in a lesion of Erythema Toxicum that has been immunostained with HMGB1 antibodies.

FIG. 16C shows a confocal micrograph of a section of the epidermal layer in a lesion of Erythema Toxicum that has been immunostained with LAMP1 antibodies.

FIG. 16D is a merged image of FIG. 16A-16C.

FIG. 16E shows a confocal micrograph of a section of the epidermal layer in a lesion of Erythema Toxicum that has been counterstained with DAPI.

FIG. 16F shows a confocal micrograph of a section of the epidermal layer in a lesion of Erythema Toxicum that has been immunostained with HMGB1 antibodies.

FIG. 16G shows a confocal micrograph of a section of the epidermal layer in a lesion of Erythema Toxicum that has been immunostained with LAMP2 antibodies.

FIG. 16H is a merged image of FIG. 16E-G.

FIG. 16I shows a confocal micrograph of a section of the epidermal layer in a lesion of Erythema Toxicum that has been counterstained with DAPI.

FIG. 16J shows a confocal micrograph of a section of the epidermal layer in a lesion of Erythema Toxicum that has been immunostained with HMGB1 antibodies.

FIG. 16K shows a confocal micrograph of a section of the epidermal layer in a lesion of Erythema Toxicum that has been immunostained with EEA1 antibodies.

FIG. 16L is a merged image of FIG. 16I-K.

FIG. 17A is the amino acid sequence of a human HMGB1 polypeptide (SEQ ID NO:1).

FIG. 17B is the amino acid sequence of a rat and mouse HMG1 polypeptide (SEQ ID NO:2).

FIG. 17C is the amino acid sequence of a human HMG2 polypeptide (SEQ ID NO:3).

FIG. 17D is the amino acid sequence of a human, mouse, and rat HMG1 A box polypeptide (SEQ ID NO:4).

FIG. 17E is the amino acid sequence of a human, mouse, and rat HMG1 B box polypeptide (SEQ ED NO:5).

FIG. 18A is the nucleic acid sequence of HMG1L5 (formerly HMG1L10; SEQ ID NO:9), which encodes an HMGB polypeptide.

FIG. 18B is the polypeptide sequence of HMG1L5 (formerly HMG1L10; SEQ ID NO:10), which is encoded by the nucleic acid sequence of FIG. 18A.

FIG. 18C is the nucleic acid sequence of HMG1L1 (SEQ ID NO:11), which encodes an HMGB polypeptide.

FIG. 18D is the polypeptide sequence of HMG1L1 (SEQ ID NO:12), which is encoded by the nucleic acid sequence of FIG. 18C.

FIG. 18E is the nucleic acid sequence of HMG1L4 (SEQ ID NO:13), which encodes an HMGB polypeptide.

FIG. 18F is the polypeptide sequence of HMG1L4 (SEQ ID NO: 14), which is encoded by the nucleic acid sequence of FIG. 18E.

FIG. 18G is the nucleic acid sequence of the BAC clone RP11-395A23 (SEQ ID NO:15), which encodes an HMG polypeptide sequence.

FIG. 18H is the amino acid sequence of the HMG polypeptide (SEQ ID NO:16) that is encoded by the BAC clone RP11-395A23 nucleic acid sequence of FIG. 18G.

FIG. 18I is the nucleic acid sequence of HMG1L9 (SEQ ID NO:17), which encodes an HMGB polypeptide.

FIG. 18J is the polypeptide sequence of HMG1L9 (SEQ ID NO:18), which is encoded by the nucleic acid sequence of FIG. 18I.

FIG. 18K is the nucleic acid sequence of LOC122441 (SEQ ID NO:19), which encodes an HMGB polypeptide.

FIG. 18L is the polypeptide sequence of LOC122441 (SEQ ID NO:20), which is encoded by the nucleic acid sequence of FIG. 18K.

FIG. 18M is the nucleic acid sequence of LOC139603 (SEQ ID NO:21), which encodes an HMGB polypeptide.

FIG. 18N is the polypeptide sequence of LOC139603 (SEQ ID NO:22), which is encoded by the nucleic acid sequence of FIG. 18M.

FIG. 18O is the nucleic acid sequence of HMG1L8 (SEQ ID NO:23), which encodes an HMGB polypeptide.

FIG. 18P is the polypeptide sequence of HMG1L8 (SEQ ID NO:24), which is encoded by the nucleic acid sequence of FIG. 18O.

### DETAILED DESCRIPTION OF THE INVENTION

The present invention is based on the discovery that antagonists of HMGB can be used to treat particular inflammatory skin conditions. In one embodiment, the HMGB antagonists used in the methods of the invention inhibit HMGB receptor binding and/or HMGB signaling. Such HMGB antagonists include, e.g., HMGB A boxes, antibodies to HMGB (e.g., antibodies to the HMGB B box, antibodies to the HMGB A box), HMGB small molecule antagonists, antibodies to TLR2, soluble TLR2 polypeptides, antibodies to RAGE, soluble RAGE polypeptides and RAGE small molecule antagonists.

A proinflammatory domain of HMGB (e.g., HMGB1) is the B box (and in particular, the first 20 amino acids of the B box), and antibodies that bind to the 3 box and inhibit proinflammatory cytokine release and inflammatory cytokine cascades can be used to alleviate deleterious symptoms caused by inflammatory cytokine cascades (PCT Publication No. WO 02/092004, the entire teachings of which are incorporated herein by reference). In addition to antibodies that bind to the B box of HMGB and inhibit proinflammatory cytokine release, antibodies that bind to the A box of HMGB can also inhibit proinflammatory cytokine release and are useful in the methods of the invention.

The A box of HMGB (e.g., HMGB1) is a weak agonist of inflammatory cytokine release, and competitively inhibits the proinflammatory activity of the B box and of HMGB (e.g., HMGB1) (PCT Publication No. WO 02/092004). Thus, HMGB A boxes (e.g., the A box of HMGB1) can be used as HMGB antagonists in the methods of the invention.

Other HMGB antagonists (e.g., inhibitors of HMGB receptor binding and/or HMGB signaling) include, e.g., antibodies to RAGE or antigen-binding fragments thereof (e.g., as taught in U.S. Pat. Nos. 5,864,018 and 5,852,174), antibodies to TLR2 or antigen-binding fragments thereof (e.g., as taught in PCT Publication Nos. WO 01/36488 and WO 00/75358), soluble RAGE, soluble TLR2 (e.g., as taught in Iwaki et al., J. Biol. Chem. 277(27):24315-24320 (2002)), HMGB small molecule antagonists (e.g., ethyl pyruvate), RAGE small molecule antagonists (e.g., as taught in PCT Publication Nos. WO 01/99210, WO 02/06965 and WO 03/075921, and U.S. Published Application No. 2002/0193432A1), TLR2 small molecule antagonists, TLR2 dominant mutant proteins, and RAGE dominant mutant proteins. Such HMGB antagonists can be used in the methods of the invention.

### HMGB Polypeptides

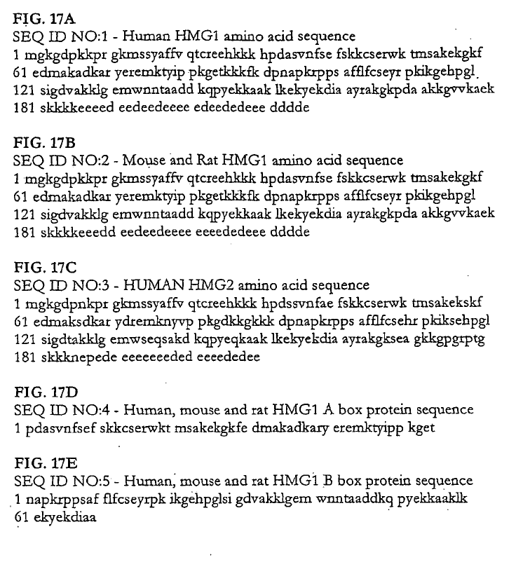

As used herein, an “HMGB polypeptide” is a polypeptide that has at least 60%, more preferably, at least 70%, 75%, 80%, 85%, or 90%, and most preferably at least 95%, sequence identity to a sequence selected from the group consisting of SEQ ID NO:1 (FIG. 17A), SEQ ID NO:2 (FIG. 17B), SEQ ID NO:3 (FIG. 17C), and SEQ ID NO:6 (MGKGDPKKPTGKMSSYAFFVQTCREEHKKKHPDASVNFSEFSKKCSERWKT MSAKEKGKFEDMAKADKARYEREMKTYIPPKGETKKKFKDPNAPKRLPSAF FLFCSEYRPKIKGEHPGLSIGDVAKKLGEMWNNTAADDKQPYEKKAAKLKE KYEKDIAAYRAKGKPDAAKKGVVKAEKSKKKKEEEEDEEDEEDEEEEEDEE DEEDEEEDDDDE) (as determined, for example, using the BLAST program and parameters described herein) and increases inflammation and/or increases release of a proinflammatory cytokine from a cell. In one embodiment, the HMGB polypeptide has one of the above biological activities. Typically, the HMGB polypeptide has both of the above biological activities.

The term “polypeptide” refers to a polymer of amino acids, and not to a specific length; thus, peptides, oligopeptides and proteins are included within the definition of a polypeptide. Preferably, the HMGB polypeptide is a mammalian HMGB polypeptide, for example, a human HMGB1 polypeptide. Preferably, the HMGB polypeptide contains a B box DNA binding domain and/or an A box DNA binding domain and/or an acidic carboxyl terminus as described herein. It is noted that the terms “HMG1” (old name) and “HMGB1” (new name) refer to the same polypeptide. Similarly, the terms “HMG2” (old name) and “HMGB2” (new name) refer to the same polypeptide.

Other examples of HMGB polypeptides are described in GenBank Accession Numbers AAA64970, AAB08987, P07155, AAA20508, S29857, P09429, NP_{—}002119, CAA31110, S02826, U00431, X67668,NP_{—}005333,NM_{—}016957, and J04179, the entire teachings of which are incorporated herein by reference. Additional examples of HMGB polypeptides include, but are not limited to, mammalian HMG1 ((HMGB1) as described, for example, in GenBank Accession Number U51677), mouse HMG1 as described, for example, in GenBank Accession Number CAA55631.1, rat HMG1 as described, for example, in GenBank Accession Number NP_{—}037095.1, cow HMG1 as described, for example, in GenBank Accession Number CAA31284.1, HMG2 ((HMGB2) as described, for example, in GenBank Accession Number M83665), HMG-2A ((HMGB3, HMG-4) as described, for example, in GenBank Accession Numbers NM_{—}005342 and NP_{—}005333), HMG14 (as described, for example, in GenBank Accession Number P05114), HMG17 (as described, for example, in GenBank Accession Number X13546), HMG1 (as described, for example, in GenBank Accession Number L17131), and HMGY (as described, for example, in GenBank Accession Number M23618); nonmammalian HMG T1 (as described, for example, in GenBank Accession Number X02666) and HMG T2 (as described, for example, in GenBank Accession Number L32859) (rainbow trout); HMG-X (as described, for example, in GenBank Accession Number D30765) (Xenopus); HMG D (as described, for example, in GenBank Accession Number X71138) and HMG Z (as described, for example, in GenBank Accession Number X71139) (Drosophila); NHP10 protein (HMG protein homolog NHP 1) (as described, for example, in GenBank Accession Number Z48008) (yeast); non-histone chromosomal protein (as described, for example, in GenBank Accession Number O00479) (yeast); HMG 1/2 like protein (as described, for example, in GenBank Accession Number Z11540) (wheat, maize, soybean); upstream binding factor (UBF-1) (as described, for example, in GenBank Accession Number X53390); PMS1 protein homolog 1 (as described, for example, in GenBank Accession Number U13695); single-strand recognition protein (SSRP, structure-specific recognition protein) (as described, for example, in GenBank Accession Number M86737); the HMG homolog TDP-1 (as described, for example, in GenBank Accession Number M74017); mammalian sex-determining region Y protein (SRY, testis-determining factor) (as described, for example, in GenBank Accession Number X53772); fungal proteins: mat-1 (as described, for example, in GenBank Accession Number AB009451), ste 11 (as described, for example, in GenBank Accession Number X53431) and Mc 1; SOX 14 (as described, for example, in GenBank Accession Number AF107043), as well as SOX 1 (as described, for example, in GenBank Accession Number Y13436), SOX 2 (as described, for example, in GenBank Accession Number Z31560), SOX 3 (as described, for example, in GenBank Accession Number X71135), SOX 6 (as described, for example, in GenBank Accession Number AF309034), SOX 8 (as described, for example, in GenBank Accession Number AF226675), SOX 10 (as described, for example, in GenBank Accession Number AJ001183), SOX 12 (as described, for example, in GenBank Accession Number X73039) and SOX 21 (as described, for example, in GenBank Accession Number AF107044); lymphoid specific factor (LEF-1) (as described, for example, in GenBank Accession Number X58636); T-cell specific transcription factor (TCF-1) (as described, for example, in GenBank Accession Number X59869); MTT1 (as described, for example, in GenBank Accession Number M62810); and SP100-HMG nuclear autoantigen (as described, for example, in GenBank Accession Number U36501). Other examples of HMGB polypeptides include those encoded by nucleic acid sequences having Genbank Accession Numbers AAH81839 (rat high mobility group box 1), NP 990233 (chicken high mobility group box 1), AAN11319 (dog high mobility group B1), AAC27653 (mole high mobility group protein), P07746 (trout high mobility group-T protein), AAA58771 (trout HMG-1), AAQ97791 (zebra fish high-mobility group box 1), AAH01063 (human high-mobility group box 2), and P10103 (cow high mobility group protein 1).

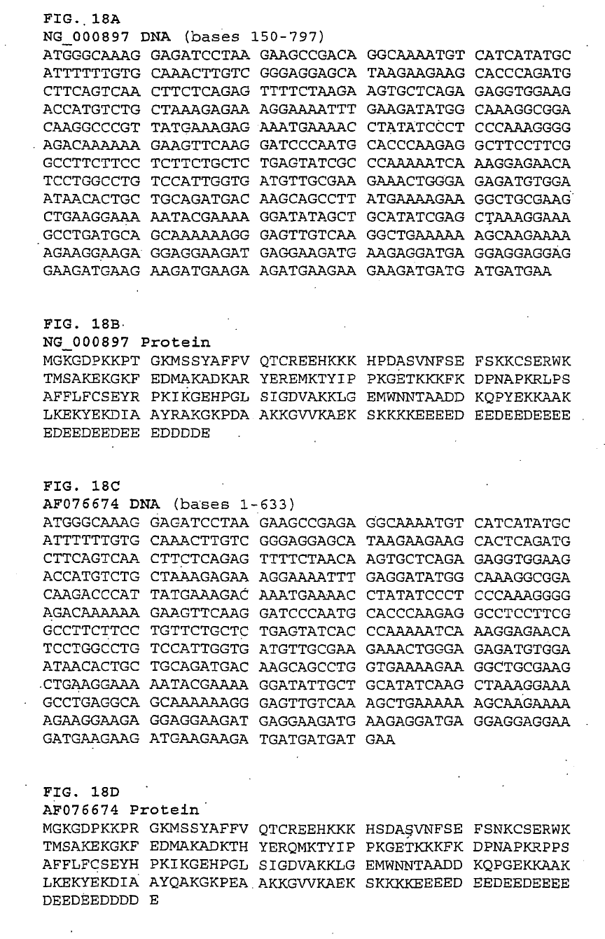

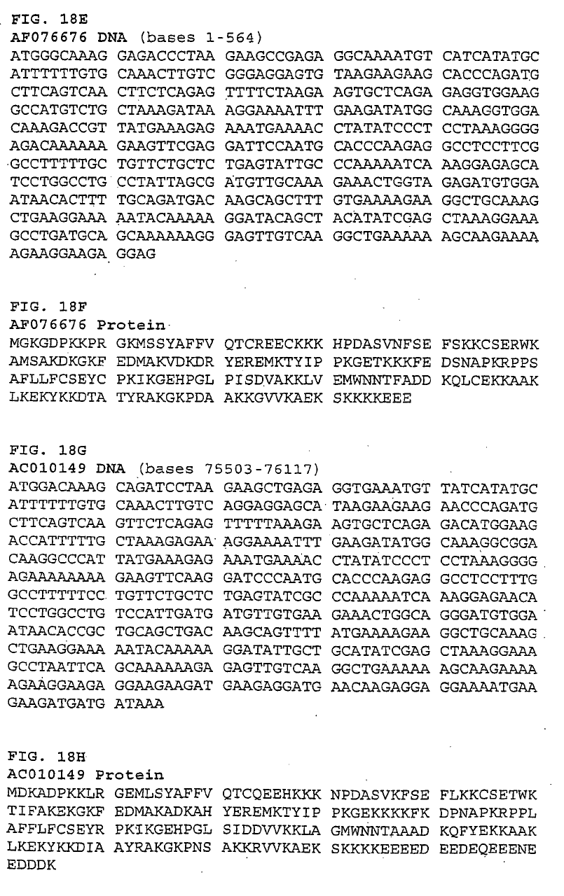

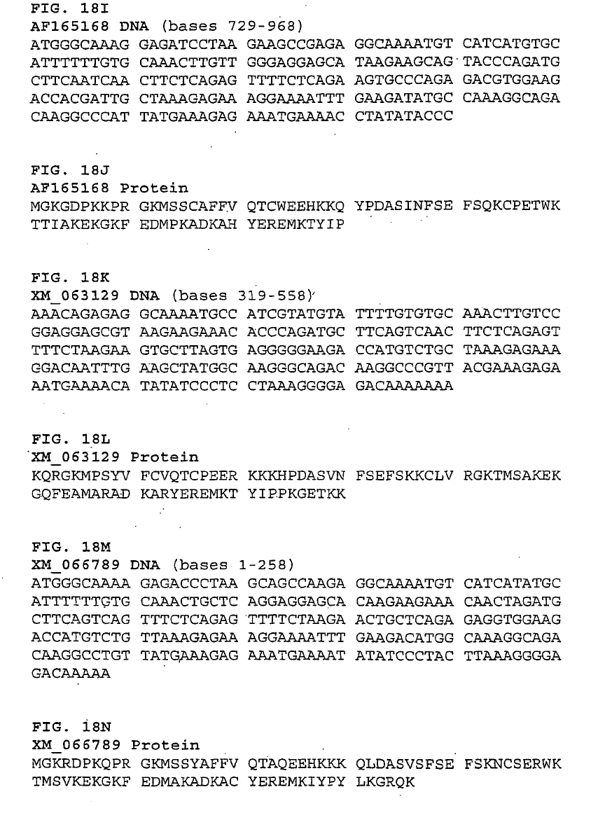

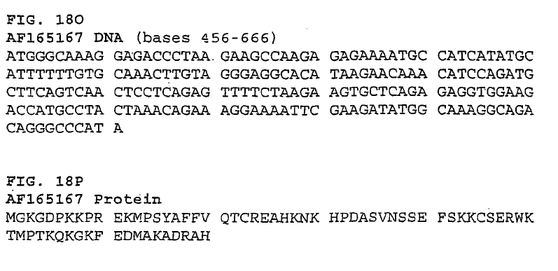

Other examples of HMGB proteins are polypeptides encoded by HMGB nucleic acid sequences having GenBank Accession Numbers NG_{—}000897 (HMG1L5) (and in particular by nucleotides 150-797 of NG_{—}000897, as shown in FIGS. 18A and 18B); AF076674 (HMG1L1) (and in particular by nucleotides 1-633 of AF076674, as shown in FIGS. 18C and 18D; AF076676 (HMG1L4) (and in particular by nucleotides 1-564 of AF076676, as shown in FIGS. 18E and 18F); AC010149 (HMG sequence from BAC clone RP11-395A23) (and in particular by nucleotides 75503-76117 of AC010149, as shown in FIGS. 18G and 18H); AF165168 (HMG1L9) (and in particular by nucleotides 729-968 of AF165168, as shown in FIGS. 18I and 18J); XM_{—}063129 (LOC122441) (and in particular by nucleotides 319-558 of XM_{—}063129, as shown in FIGS. 18K and 18L); XM_{—}066789 (LOC139603) (and in particular by nucleotides 1-258 of XM_{—}066789, as shown in FIGS. 18M and 18N); and AF165167 (HMG1L8) (and in particular by nucleotides 456-666 of AF165167, as shown in FIGS. 18O and 18P).

Optionally, the HMGB polypeptide is a substantially pure, or substantially pure and isolated, polypeptide that has been separated from components that naturally accompany it. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell (e.g., in a “fusion protein”) and still be “isolated” or “purified.” It is understood, however, that preparations in which the polypeptide is not purified to homogeneity are useful. For example, the polypeptide may be in an unpurified form, for example, in a cell, cell milieu, or cell extract. The critical feature is that the preparation allows for the desired function of the polypeptide, even in the presence of considerable amounts of other components.

An HMGB polypeptide can be purified from cells that naturally express it, from cells that have been altered to express it (recombinant), or synthesized using known protein synthesis methods. In one embodiment, the polypeptide is produced by recombinant DNA techniques. For example, a nucleic acid molecule encoding the polypeptide is cloned into an expression vector, the expression vector is introduced into a host cell and the polypeptide is expressed in the host cell. The polypeptide can then be isolated from the cells by an appropriate purification scheme using standard protein purification techniques.

Functional equivalents of HMGB (proteins or polypeptides that have one or more of the biological activities of an HMGB polypeptide) can also be used in the methods of the present invention. Biologically active fragments, sequence variants, post-translationally modified proteins, and chimeric or fusion proteins comprising an HMGB polypeptide, a biologically active fragment or a variant are examples of functional equivalents of HMGB. Variants include a substantially homologous polypeptide encoded by the same genetic locus in an organism, i.e., an allelic variant, as well as other splicing variants. Variants also encompass polypeptides derived from other genetic loci in an organism, but having substantial homology to the protein of interest, for example, an HMGB protein as described herein.

A variant polypeptide can differ in amino acid sequence by one or more substitutions, deletions, insertions, inversions, fusions, and truncations, or a combination of any of these. Further, variant polypeptides can be fully functional or can lack function in one or more activities. Fully functional variants typically contain only conservative variations or variations in non-critical residues or in non-critical regions. Functional variants can also contain substitution of similar amino acids that result in no change or an insignificant change in function. Alternatively, such substitutions may positively or negatively affect function to some degree.

Amino acids that are essential for function can be identified by methods known in the art, such as site-directed mutagenesis or alanine-scanning mutagenesis (Cunningham et al., Science, 244:1081-1085 (1989)). The latter procedure introduces single alanine mutations at every residue in the molecule. The resulting mutant molecules are then tested for biological activity in vitro. Sites that are critical for polypeptide activity can also be determined by structural analysis, e.g., by crystallization, nuclear magnetic resonance and/or photoaffinity labeling (Smith et al., J. Mol. Biol., 224:899-904 (1992); and de Vos et al., Science, 255:306-312 (1992)).

HMGB functional equivalents also include polypeptide fragments of HMGB. Fragments can be derived from an HMGB polypeptide or HMGB variant. As used herein, a fragment comprises at least 6 contiguous amino acids from an HMGB polypeptide. Useful fragments include those that retain one or more of the biological activities of the polypeptide. Examples of HMGB biologically active fragments include the B box, as well as biologically active fragments of the B box, for example, the first 20 amino acids of the B box (e.g., the first 20 amino acids of SEQ ID NO:5 (SEQ ID NO:44; NAPKRPPSAFFLFCSEYRPK) or SEQ ID NO:8 (SEQ ID NO:45; FKDPNAPKRLPSAFFLFCSE)). Other examples of HMGB biologically active fragments include the A box, as well as biologically active fragments of the A box.

Biologically active fragments (peptides which are, for example, 6, 9, 12, 15, 16, 20, 30, 35, 36, 37, 38, 39, 40, 50 or 100 or more amino acids in length) can comprise a domain, segment, or motif that has been identified by analysis of the polypeptide sequence using well-known methods, e.g., signal peptides, extracellular domains, one or more transmembrane segments or loops, ligand binding regions, zinc finger domains, DNA binding domains, or post-translation modification sites. Example of domains include the A box and B box, as described herein.

Fragments call be discrete (not fused to other amino acids or polypeptides) or can be within a larger polypeptide. Further, several fragments can be comprised within a single larger polypeptide. In one embodiment, a fragment designed for expression in a host can have heterologous pre- and pro-polypeptide regions fused to the amino terminus of the polypeptide fragment and an additional region fused to the carboxyl terminus of the fragment.

The invention also provides uses and methods for chimeric or fusion polypeptides containing an HMGB polypeptide or a functional equivalent of HMGB. These chimeric proteins comprise an HMGB polypeptide or fragment thereof operatively linked to a heterologous protein or polypeptide having an amino acid sequence not substantially homologous to the polypeptide. “Operatively linked” indicates that the polypeptide and the heterologous protein are fused in-frame. The heterologous protein can be fused to the N-terminus or C-terminus of the polypeptide. In one embodiment the fusion polypeptide does not affect function of the HMGB polypeptide per se. For example, the fusion polypeptide can be a Glutathione S-transferase (GST)-fusion polypeptide in which the polypeptide sequences are fused to the C-terminus of a GST sequence. Other types of fusion polypeptides include, but are not limited to, enzymatic fusion polypeptides, for example, β-galactosidase fusion polypeptides, yeast two-hybrid GAL fusion polypeptides, poly-His fusions, FLAG-tagged fusion polypeptides, GFP fusion polypeptides, and Ig fusion polypeptides. Such fusion polypeptides can facilitate the purification of recombinant polypeptide. In certain host cells (e.g., mammalian host cells), expression and/or secretion of a polypeptide can be increased by using a heterologous signal sequence. Therefore, in another embodiment, the fusion polypeptide contains a heterologous signal sequence at its N-terminus.

EP-A-O 464 533 discloses fusion proteins comprising various portions of immunoglobulin constant regions. The Fe is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists (Bennett et al., Journal of Molecular Recognition 8:52-58 (1995); and Johanson et al., J. Biol. Chem., 270(16):9459-9471 (1995)). Thus, this invention also encompasses soluble fusion polypeptides containing a polypeptide of the invention and various portions of the constant regions of heavy or light chains of immunoglobulins of various subclass (e.g., IgG, IgM, IgA, IgE).

A chimeric or fusion polypeptide can be produced by standard recombinant DNA techniques. For example, DNA fragments coding for the different polypeptide sequences (e.g., an HMGB polypeptide and another polypeptide) are ligated together in-frame in accordance with conventional techniques. In another embodiment, the fusion gene can be synthesized by conventional techniques, e.g., using an automated DNA synthesizer. Alternatively, PCR amplification of nucleic acid fragments can be carried out using anchor primers that give rise to complementary overhangs between two consecutive nucleic acid fragments that can subsequently be annealed and re-amplified to generate a chimeric nucleic acid sequence (see Ausubel et al., Current Protocols in Molecular Biology, 1992). Moreover, many expression vectors are commercially available that already encode a fusion moiety (e.g., a GST moiety). A nucleic acid molecule encoding an HMGB polypeptide can be cloned into such an expression vector, such that the fusion moiety is linked in-frame to the HMGB polypeptide.

HMGB functional equivalents can be generated using standard molecular biology techniques and assaying the function using, for example, methods described herein, such as, determining if the functional equivalent, when administered to a cell (e.g., a macrophage), increases release of a proinflammatory cytokine from the cell, as compared to an untreated control cell. In one embodiment, the HMGB functional equivalent has at least 50%, 60%, 70%, 80%, or 90% of the biological activity of the HMGB1 polypeptide of SEQ ID NO:1.

### HMGB A Boxes

In particular embodiments, the methods of the present invention employ HMGB A boxes as HMGB antagonists. As used herein, an “HMGB A box”, also referred to herein as an “A box” (and also known as HMG A box), is a protein or polypeptide that has at least 50%, 60%, 70%, 75%, 80%, 85%, 90%, or 95%, sequence identity to an HMGB A box as described herein, and has one or more of the following biological activities: inhibiting inflammation mediated by HMGB and/or inhibiting release of a proinflaimmatory cytokine from a cell. In one embodiment, the HMGB A box polypeptide has one of the above biological activities. Typically, the HMGB A box polypeptide has both of the above biological activities. In one embodiment, the A box has at least 50%, 60%, 70%, 75%, 80%, 85%, 90%, or 95%, sequence identity to SEQ ID NO:4 (FIG. 17D) and/or SEQ ID NO:7 (PTGKMSSYAF FVQTCREEHK KKHPDASVNF SEFSKKCSER WKTMSAKEKG KFEDMAKADK ARYEREMKTY IPPKGET (SEQ ID NO:7)). In other embodiments, the HMGB A box has no more than 10%, 20%, 25%, 30%, 40%, 50%, 60%, 70%, 80%, or 90%, of the biological activity of full length HMGB. In another embodiment, the HMGB A box amino acid consists of the sequence of SEQ ID NO:4 (FIG. 17D) or SEQ ID NO:7, or the amino acid sequence in the corresponding region of an HMGB protein in a mammal. An HMGB A box is also a recombinantly-produced polypeptide having the same amino acid sequence as the A box sequences described above. The HMGB A box is preferably a vertebrate HMGB A box, for example, a mammalian HMGB A box, more preferably, a mammalian HMBG1 A box, for example, ahuman HMGB1 A box, and most preferably, the HMGB1 A box comprising, or consisting of, the sequence of SEQ ID NO:4 (FIG. 17D) or SEQ ID NO:7.

An HMGB A box often has no more than about 85 amino acids and no fewer than about 4 amino acids. Examples of polypeptides having A box sequences within them include, but are not limited to, the HMGB polypeptides described herein. The A box sequences in such polypeptides can be determined and isolated using methods described herein, for example, by sequence comparisons to A boxes described herein and testing for biological activity using methods described herein and/or other method known in the art.

In addition to A boxes that can be found in the HMGB polypeptides described herein, other HMGB A box polypeptide sequences include the following sequences:

(human HMGB1; SEQ ID NO: 25)

PDASVNFSEF SKKCSERWKT MSAKEKGKFE DMAKADKARY
EREMKTYIPP KGET;

(human HMGB2; SEQ ID NO: 26)

DSSVNFAEF SKKCSERWKT MSAKEKSKFE DMAKSDKARY
DREMKNYVPP KGDK;

(human HMGB3; SEQ ID NO: 27)

PEVPVNFAEF SKKCSERWKT VSGKEKSKFD EMAKADKVRY
DREMKDYGPA KGGK;

(HMG1L5; SEQ ID NO: 28)

PDASVNFSEF SKKCSERWKT MSAKEKGKFE DMAKADKARY
EREMKTYIPP KGET;

(HMG1L1; SEQ ID NO: 29)

SDASVNFSEF SNKCSERWKT MSAKEKGKFE DMAKADKTHY
ERQMKTYIPP KGET;

(HMG1L4; SEQ ID NO: 30)

PDASVNFSEF SKKCSERWKA MSAKDKGKFE DMAKVDKADY
EREMKTYIPP KGET;

(HMG sequence from BAC clone
RP11-395A23; SEQ ID NO: 31)

PDASVKFSEF LKKCSETWKT IFAKEKGKFE DMAKADKAHY
EREMKTYIPP KGEK;

(HMG1L9; SEQ ID NO: 32)

PDASINFSEF SQKCPETWKT TIAKEKGKFE DMAKADKAHY
EREMKTYTPP KGET;

(HMG1L8; SEQ ID NO: 33)

PDASVNSSEF SKKCSERWKT MPTKQGKFED MAKADRAH;

(LOC122441; SEQ ID NO: 34)

PDASVNFSEF SKKCLVRGKT MSAKEKGQFE AMARADKARY
EREMKTYIP PKGET;

(LOC139603; SEQ ID NO: 35)

LDASVSFSEF SNKCSERWKT MSVKEKGKFE DMAKADKACY
EREMKIYPYL KGRQ;
and

(human HMGB1 A box; SEQ ID NO: 36)

GKGDPKKPRG KMSSYAFFVQ TCREEHKKKH PDASVNFSEF
SKKCSERWKT MSAKEKGKFE DMAKADKARY EREMKTYIPP
KGET.

Functional equivalents of HMGB A boxes can also be used in the methods of the present invention. In one embodiment, a functional equivalent of an HMGB A box inhibits release of a proinflammatory cytokine from a cell treated with an HMGB polypeptide. Examples of HMGB A box functional equivalents include, for example, biologically active fragments, post-translational modifications, variants, or fusion proteins comprising A boxes, as defined herein. A box functional equivalents can be generated using standard molecular biology techniques and assaying the function using known methods, for example, by determining if the functional equivalent (e.g., fragment), when administered to a cell (e.g., a macrophage), decreases or inhibits release of a proinflammatory cytokine from the cell. In one embodiment, the A box functional equivalent has at least 50%, 60%, 70%, 80%, or 90%, of the biological activity of the HMGB1 polypeptide of SEQ ID NO:4.

Optionally, the HMGB A box polypeptide is a substantially pure, or substantially pure and isolated, polypeptide that has been separated from components that naturally accompany it. The polypeptide may also be in an unpurified form, for example, in a cell, cell milieu, or cell extract. The critical feature is that the preparation allows for the desired function of the HMGB A box polypeptide, even in the presence of considerable amounts of other components.

As described herein, in particular embodiments, the methods of the invention utilize antibodies to the HMGB A box or antigen-binding fragments thereof.

### HMGB B Boxes

In certain embodiments, the methods of the present invention employ antibodies to the HMGB B box or antigen-binding fragments thereof. As used herein, an “HMGB B box”, also referred to herein as a “B box” (and also known as an HMG B box), is a polypeptide that has at least 60%, 65%, 70%, 75%, 80%, 85%, 90%, or 95%, sequence identity to SEQ ID NO:5 (FIG. 17E) and/or SEQ ID NO:8 (FKDPNAPKRL PSAFFLFCSE YRPKIKGEHP GLSIGDVAKK LGEMWNNTAA DDKQPYEKKA AKLKEKYEKD IAAY (SEQ ID NO:8)) (as determined using the BLAST program and parameters described herein), lacks an A box, and has one or more of the following biological activities: increasing inflammation and/or increasing release of a proinflammatory cytokine from a cell. In one embodiment, the HMGB B box polypeptide has one of the above biological activities. Typically, the HMGB B box polypeptide has both of the above biological activities. Preferably, the HMGB B box has at least 25%, 30%, 40%, 50%, 60%, 70%, 80%, or 90%, of the biological activity of full length HMGB. In another embodiment, the HMGB box comprises, or consists of, the sequence of SEQ ID NO:5 or SEQ ID NO:8, or the amino acid sequence in the corresponding region of an HMGB protein in a mammal.

Preferably, the HMGB B box is a mammalian EMGB B box, for example, a human HMGB1 B box. An HMGB B box often has no more than about 85 amino acids and no fewer than about 4 amino acids. Examples of polypeptides having B box sequences within them include, but are not limited to, the HMGB polypeptides described herein. The B box sequences in such polypeptides can be determined and isolated using methods described herein, for example, by sequence comparisons to B boxes described herein and testing for biological activity using methods described herein and/or other method known in the art.

In addition to B boxes that can be found in the HMGB polypeptides described herein, other HMGB B box polypeptide sequences include the following sequences:

(human HMGB1; SEQ ID NO: 37)

FKDPNAPKRP PSAFFLFCSE YRPKIKGEHP GLSIGDVAKK
LGEMWNNTAA DDKQPYEKKA AKLKEKYEKD IAAY;

(human HMGB2; SEQ ID NO: 38)

KKDPNAPKRP PSAFFLFCSE HRPKIKSEHP GLSIGDTAKK
LGEMWSEQSA KDKQPYEQKA AKLKEKYEKD IAAY;

(HMG1L5; SEQ ID NO: 39)

FKDPNAPKRL PSAFFLFCSE YRPKIKGEHP GLSIGDVAKK
LGEMWNNTAA DDKQPYEKKA AKLKEKYEKD IAAY;

(HMG1L1; SEQ ID NO: 40)

FKDPNAPKRP PSAFFLFCSE YHPKIKGEHP GLSIGDVAKK
LGEMWNNTAA DDKQPGEKKA AKLKEKYEKD IAAY;

(HMG1L4; SEQ ID NO: 41)

FKDSNAPKRP PSAFLLFCSE YCPKIKGEHP GLPISDVAKK
LVEMWNNTFA DDKQLCEKKA AKLKEKYKKD TATY;

(HMG sequence from BAC clone
RP11-359A23; SEQ ID NO: 42)

FKDPNAPKRP PSAFFLFCSE YRPKIKGEHP GLSIGDVVKK
LAGMWNNTAA ADKQFYEKKA AKLKEKYKKD IAAY;
and

(human HMGB1 box; SEQ ID NO: 43)

FKDPNAPKRP PSAFFLFCSE YRPKIKGEHP GLSIGDVAKK
LGEMWNNTAA DDKQPYEKKA AKLKEKYEKD IAAYRAKGKP
DAAKKGVVKA EK.

Antibodies to functional equivalents of HMGB B boxes can also be used in the methods of the present invention. Examples of HMGB B box functional equivalents include, for example, biologically active fragments, post-translational modifications, variants, or fusion proteins comprising B boxes, as defined herein. B box functional equivalents can be generated using standard molecular biology techniques and assaying the function using known methods, for example, by determining if the functional equivalent (e.g., fragment), when administered to a cell (e.g., a macrophage) increases release of a proinflammatory cytokine from the cell. In one embodiment, the B box functional equivalent has at least 50%, 60%, 70%, 80%, or 90%, of the biological activity of the B box polypeptide of SEQ ID NO:5 (FIG. 17E). Preferred examples of B box biological equivalents are polypeptides comprising, or consisting of, the first 20 amino acids of the B box (e.g., the first 20 amino acids of SEQ ID NO:5 (i.e., SEQ ID NO:44; NAPKRPPSAFFLFCSEYRPK) or the first 20 amino acids of SEQ ID NO:8 (i.e., SEQ ID NO:45; FKDPNAPKRLPSAFFLFCSE)).

Optionally, the HMGB B box polypeptide is a substantially pure, or substantially pure and isolated, polypeptide that has been separated from components that naturally accompany it. Alternatively, the polypeptide may be in an unpurified form, for example, in a cell, cell milieu, or cell extract. The critical feature is that the preparation allows for the desired function of the polypeptide, even in the presence of considerable amounts of other components.

HMGB, HMGB A box, and/or HMGB B box, functional equivalents, either naturally occurring or non-naturally occurring, include polypeptides that have sequence identity to the HMGB polypeptides, HMGB A boxes, and HMGB B boxes described herein. As used herein, two polypeptides (or regions of the polypeptides) are substantially homologous or identical when the amino acid sequences are at least about 60%, 70%, 75%, 80%, 85%, 90%, or 95% or more, homologous or identical. The percent identity of two amino acid sequences (or two nucleic acid sequences) can be determined by aligning the sequences for optimal comparison purposes (e.g., gaps can be introduced into one or both of the sequences). The amino acids or nucleotides at corresponding positions are then compared, and the percent identity between the two sequences is a function of the number of identical positions shared by the sequences (i.e., % identity=# of identical positions/total # of positions×100). In certain embodiments, the length of the HMGB polypeptide, HMGB A box polypeptide, or HMGB B box polypeptide aligned for comparison purposes is at least 30%, preferably at least 40%, more preferably at least 60%, and even more preferably at least 70%, 80%, 90%, or 100%, of the length of the reference sequence, for example, those sequences provided in FIGS. 17A-17E, FIGS. 18A-18P, and SEQ ID NOS:25-43. The actual comparison of the two sequences can be accomplished by well-known methods, for example, using a mathematical algorithm. A preferred, non-limiting example of such a mathematical algorithm is described in Karlin et al. (Proc. Natl. Acad. Sci. USA, 90:5873-5877 (1993)). Such an algorithm is incorporated into the BLASTN and BLASTX programs (version 2.2) as described in Schaffer et al. (Nucleic Acids Res., 29:2994-3005 (2001)). When utilizing BLAST and Gapped BLAST programs, the default parameters of the respective programs (e.g., BLASTN; available at the Internet site for the National Center for Biotechnology Information) can be used. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Another preferred, non-limiting example of a mathematical algorithm utilized for the comparison of sequences is the algorithm of Myers and Miller, CABIOS (1989). Such an algorithm is incorporated into the ALIGN program (version 2.0), which is part of the GCG (Accefrys, San Diego, Calif.) sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (Comput. Appl. Biosci., 10:3-5 (1994)); and FASTA described in Pearson and Lipman (Proc. Natl. Acad. Sci USA, 85:2444-2448 (1988)).

In another embodiment, the percent identity between two amino acid sequences can be accomplished using the GAP program in the GCG software package (Accelrys, San Diego, Calif.) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package (Accelrys, San Diego, Calif.), using a gap weight of 50 and a length weight of 3.

### Antibodies to HMGB, HMGB B Box and HMGB A Box Polypeptides

The present invention is directed in part to methods utilizing antibodies and antigen-binding fragments thereof that bind to an HMGB polypeptide or a biologically active fragment thereof (anti-HMGB antibodies). The anti-HMGB antibodies and antigen-binding fragments can be neutralizing antibodies or antigen-binding fragments (i.e., they can inhibit a biological activity of an HMG polypeptide or a fragment thereof, for example, the release of a proinflammatory cytokine from a vertebrate cell induced by HMGB). The invention encompasses antibodies and antigen-binding fragments that selectively bind to an HMGB B box or a fragment thereof, but do not selectively bind to non-B box epitopes of HMGB (anti-HMGB B box antibodies and antigen-binding fragments thereof). The invention further encompasses antibodies and antigen-binding fragments that selectively bind to an HMGB A box or a functional equivalent thereof, but do not selectively bind to non-A box epitopes of HMGB (anti-HMGB A box antibodies and antigen-binding fragments thereof). In these embodiments, the antibodies and antigen-binding fragments can also be neutralizing antibodies and antigen-binding fragments (i.e., they can inhibit a biological activity of an HMGB polypeptide or a B box polypeptide or fragment thereof, for example, the release of a proinflammatory cytokine from a vertebrate cell induced by HMGB). Antibodies to HMGB have been shown to inhibit release of a proinflammatory cytokine from a cell treated with an HMGB polypeptide (see, for example, PCT publication WO 02/092004). Such antibodies can be used in the methods of the invention.

The term “antibody” or “purified antibody” as used herein refers to immunoglobulin molecules. The term “antigen-binding fragment” or “purified antigen-binding fragment” as used herein refers to immunologically active portions of immunoglobulin-molecules, i.e., molecules that contain an antigen binding site that selectively bind to an antigen. A molecule that selectively binds to a polypeptide of the invention is a molecule that binds to that polypeptide or a fragment thereof, but does not substantially bind other molecules in a sample, e.g., a biological sample that naturally contains the polypeptide. Preferably the antibody is at least 60%, by weight, free from proteins and naturally occurring organic molecules with which it naturally associates. More preferably, the antibody preparation is at least 75%, or 90%, and most preferably at least 99%, by weight, antibody. Examples of immunologically active portions of immunoglobulin molecules include, but are not limited to Fv, Fab, Fab′ and F(ab′)₂ fragments. Such fragments can be produced by enzymatic cleavage or by recombinant techniques. For example, papain or pepsin cleavage can generate Fab or F(ab′)₂ fragments, respectively. Other proteases with, the requisite substrate specificity can also be used to generate Fab or F(ab′)₂ fragments. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH₁ domain and hinge region of the heavy chain.

The invention provides polyclonal and monoclonal antibodies that selectively bind to an HMGB polypeptide, an HMGB B box polypeptide, and/or an HMGB A box polypeptide. The term “monoclonal antibody” or “monoclonal antibody composition,” as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared, e.g., by immunizing a suitable subject with a desired immunogen, e.g., an HMGB polypeptide, an HMGB B box polypeptide, an HMGB A box polypeptide or fragments thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction.

At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (Nature 256:495-497 (1975)), the human B cell hybridoma technique (Kozbor et al., Immunol. Today 4:72 (1983)), the EBV-hybridoma technique (Cole et al., Monoclonal Antibodies and Cancer Therapy, Alan R. Liss, Inc., pp. 77-96 (1985)) or trioma techniques. The technology for producing hybridomas is well known (see generally Current Protocols in 25 Immunology, Coligan et al., (eds.) John Wiley & Sons, Inc., New York, N.Y. (1994)). Briefly, an immortal cell line (typically a myeloma) is fused to lymphocytes (typically splenocytes) from a mammal immunized with an immunogen, and the culture supernatants of the resulting hybridoma cells are screened to identify a hybridoma producing a monoclonal antibody that binds to the desired polypeptide (e.g., an HMGB polypeptide, an HMGB B box polypeptide, an HMGB A box polypeptide).

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature, 266:55052, 1977; R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

In one alternative to preparing monoclonal antibody-secreting hybridomas, a monoclonal antibody to an HMGB polypeptide, an HMGB B box polypeptide and/or an HMGB A box polypeptide, can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9:1370-1372 (1991); Hay et al., Hum. Antibod. Hybridomas 3:81-85 (1992); Huse et al., Science 246:1275-1281 (1989); and Griffiths et al., EMBO J. 12:725-734 (1993). Phage display technology can also be utilized to select antibody genes with binding activities towards an HMGB polypeptide either from repertoires of PCR amplified v-genes of lymphocytes from humans screened for possessing anti-B box antibodies or from naive libraries (McCafferty et al., Nature 348:552-554, 1990; and Marks, et al., Biotechnology 10:779-783, 1992). The affinity of these antibodies can also be improved by chain shuffling (Clackson et al., Nature 352: 624-628, 1991).

Single chain antibodies, and recombinant antibodies, such as chimeric, humanized, primratized (CDR-grafted) or veneered antibodies, as well as chimeric, CDR-grafted or veneered single chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single chain antibodies. Techniques for the production of single chain antibodies (U.S. Pat. No.4,946,778) can be adapted to produce single chain antibodies to the HMGB polypeptides or HMGB B box polypeptides or fragments thereof. Also, transgenic mice, or other organisms such as other mammals, may be used to express humanized antibodies.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. In one embodiment, cloned variable regions can be mutated, and sequences encoding variants with the desired specificity can be selected (e.g., from a phage library; see e.g., Krebber et al., U.S. Pat. No. 5,514,548; Hoogenboom et al., WO 93/06213).

If the antibody is used therapeutically in in vivo applications, the antibody can be modified to make it less immunogenic. For example, if the individual is human the antibody is preferably “humanized”; where the complementarity determining region(s) (CDRs) of the antibody is transplanted into a human antibody (for example, as described in Jones et al., Nature 321:522-525, 1986; and Tempest et al., Biotechnology 9:266-273 (1991)). The antibody can be a humanized antibody comprising one or more immunoglobulin chains, said antibody comprising a CDR of nonhuman origin (e.g., one or more CDRs derived from an antibody of nonhuman origin) and a framework region derived from a light and/or heavy chain of human origin (e.g., CDR-grafted antibodies with or without framework changes). In one embodiment, the antibody or antigen-binding fragment thereof comprises the light chain CDRs (CDR1, CDR2 and CDR3) and heavy chain CDRs (CDR1, CDR2 and CDR3) of a particular immunoglobulin. In another embodiment, the antibody or antigen-binding fragment further comprises a human framework region.

Human antibodies and nucleic acids encoding the same can be obtained from a human or from human-antibody transgenic animals. Human-antibody transgenic animals (e.g., mice) are animals that are capable of producing a repertoire of human antibodies, such as XENOMOUSE (Abgenix, Fremont, Calif.), HUMAB-MOUSE, KIRIN TC MOUSE or KM-MOUSE (MEDAREX, Princeton, N.J.). Generally, the genome of human-antibody transgenic animals has been altered to include a transgene comprising DNA from a human immunoglobulin locus that can undergo functional rearrangement. An endogenous immunoglobulin locus in a human-antibody transgenic animal can be disrupted or deleted to eliminate the capacity of the animal to produce antibodies encoded by an endogenous gene. Suitable methods for producing human-antibody transgenic animals are well known in the art. (See, for example, U.S. Pat. Nos. 5,939,598 and 6,075,181 (Kucherlapati et al.), U.S. Pat. Nos. 5,569,825, 5,545,806, 5,625,126, 5,633,425, 5,661,016, and 5,789,650 (Lonberg et al.), Jakobovits et al., Proc. Natl. Acad. Sci. USA, 90: 2551-2555 (1993), Jakobovits et al., Nature, 362: 255-258 (1993), Jakobovits et al. WO 98/50433, Jakobovits et al. WO 98/24893, Lonberg et al. WO 98/24884, Lonberg et al. WO 97/13852, Lonberg et al. WO 94/25585, Lonberg et al. EP 0 814 259 A2, Lonberg et al. GB 2 272 440 A, Lonberg et al., Nature 368:856-859 (1994), Lonberg et al., Int Rev Immunol 13(1):65-93 (1995), Kucherlapati et al. WO 96/34096, Kucherlapati et al. EP 0 463 151 B1, Kucherlapati et al. EP 0 710 719 A1, Surani et al. U.S. Pat. No. 5,545,807, Bruggemann et al. WO 90/04036, Bruggemanm et al. EP 0 438 474 B1, Taylor et al., Int. Immunol. 6(4)579-591 (1994), Taylor et al., Nucleic Acids Research 20(23):6287-6295 (1992), Green et al., Nature Genetics 7:13-21 (1994), Mendez et al., Nature Genetics 15:146-156 (1997), Tuaillon et al., Proc Natl Acad Sci USA 90(8):3720-3724 (1993) and Fishwild et al., Nat Biotechnol 14(7):845-851 (1996), the teachings of each of the foregoing are incorporated herein by reference in their entirety.)

Because vertebrate HMGB polypeptides, HMGB B boxes and HMGB A boxes show a high degree of sequence conservation, it is reasonable to believe that antibodies that bind to vertebrate HMGB polypeptides, HMGB B boxes or HMGB A boxes in general can induce release of a proinflammatory cytokine from a vertebrate cell. Therefore, antibodies against vertebrate HMGB polypeptides or HMGB B boxes without limitation are within the scope of the invention.

When the antibodies are obtained that specifically bind to HMGB epitopes, HMGB B box epitopes and/or HMGB A box epitopes, they can then be screened without undue experimentation for the ability to inhibit release of a proinflammatory cytokine using standard methods. Anti-HMGB antibodies, anti-HMGB B box antibodies and anti-HMGB A box antibodies that can inhibit the production of any single proinflammatory cytokine, and/or inhibit the release of a proinflammatory cytokine from a cell, and/or inhibit a condition characterized by activation of an inflammatory cytokine cascade are within the scope of the present invention. Preferably, the antibodies can inhibit the production of TNF (e.g., TNF-α), IL-1β, or IL-6.

Polyclonal antibodies raised against HMGB have been produced (see, for example, U.S. Pat. No. 6,468,555 B1, the entire teachings of which are incorporated herein by reference). These antibodies have been shown to inhibit release of a proinflammatory cytokine from a cell, and to treat inflammation.

Polyclonal antibodies against the HMGB1 B box have also been produced (see, for example, PCT Publication No. WO 02/092004). Such antibodies detected full length HMGB1 and HMGB1 B box in immunoassays, but did not cross react with TNF, IL-1 or IL-6. These HMGB1 B box antibodies also inhibited release of a proinflammatory cytokine from a cell and provided protection against sepsis induced by cecal ligation and puncture.

Monoclonal antibodies to HMGB1 are known in the art, and are taught, for example, in WO 2005/026209; the entire teachings of which are incorporated herein by reference. Particular monoclonal antibodies to HMGB1 include, e.g., 6E6 HMGB1 mAb, 2E11 HMGB1 mAb, 6H9 HMGB1 mAb, 10D4 HMGB1 mAb and 2G7 HMGB1 mAb.

6E6 HMGB1 mAb, also referred to as 6E6-7-1-1 or 6E6, can be produced by murine hybridoma 6E6 HMGB1 mAb, which was deposited on Sep. 3, 2003, on behalf of Critical Therapeutics, Inc., 675 Massachusetts Avenue, 14^{th} Floor, Cambridge, Mass. 02139, U.S.A., at the American Type Culture Collection, 10801 University Boulevard, Manassas, Va. 20110, U.S.A., under Accession No. PTA-5433.

2E11 HMGB1 mAb, also referred to as 2E 1-1-1-2 or 2E11, can be produced by murine hybridoma 2E11 HMGB1 mAb, which was deposited on Sep. 3, 2003, on behalf of Critical Therapeutics, Inc., 675 Massachusetts Avenue, 14^{th} Floor, Cambridge, Mass. 02139, U.S.A., at the American Type Culture Collection, 10801 University Boulevard, Manassas, Va. 20110, U.S.A., under Accession No. PTA-5431.

6H9 HMGB1 mAb, also referred to as 6H9-1-1-2 or 6H9, can be produced by murine hybridoma 6H9 HMGB1 mAb, which was deposited on Sep. 3, 2003, on behalf of Critical Therapeutics, Inc., 675 Massachusetts Avenue, 14^{th} Floor, Cambridge, Mass. 02139, U.S.A., at the American Type Culture Collection, 10801 University Boulevard, Manassas, Va. 20110, U.S.A., under Accession No. PTA-5434.

10D4 HMGB1 mAb, also referred to as 10D4-1-1-1-2 or 10D4, can be produced by murine hybridoma 10D4 HMGB1 mAb, which was deposited on Sep. 3, 2003, on behalf of Critical Therapeutics, Inc., 675 Massachusetts Avenue, 14^{th} Floor, Cambridge, Mass. 02139, U.S.A., at the American Type Culture Collection, 10801 University Boulevard, Manassas, Va. 20110, U.S.A., under Accession No. PTA-5435.

2G7 HMGB1 mAb, also referred to as 3-2G7-1-1-1 or 2G7, can be produced by murine hybridoma 2G7 HMGB1 mAb, which was deposited on Sep. 3, 2003, on behalf of Critical Therapeutics, Inc., 675 Massachusetts Avenue, 14^{th} Floor, Cambridge, Mass. 02139, U.S.A., at the American Type Culture Collection, 10801 University Boulevard, Manassas, Va. 20110, U.S.A., under Accession No. PTA-5432.

As described herein, in certain embodiments the methods of the invention utilize antibodies or antigen-binding fragments thereof, that bind an HMGB polypeptide or fragment thereof (e.g., an HMGB B box or a biologically active fragment thereof, an HMGB A box or a biologically active fragment thereof). Such HMGB polypeptides include, e.g., the HMGB polypeptides described herein. In one embodiment, the antibody or antigen-binding fragment binds a mammalian HMGB polypeptide, a mammalian HMGB B Box polypeptide and/or a mammalian HMGB A Box polypeptide. In another embodiment, the antibody or antigen-binding fragment binds an HMGB1 polypeptide, an HMGB1 B Box polypeptide and/or a mammalian HMGB A Box polypeptide. In yet another embodiment, the antibody or antigen-binding fragment binds an HMGB1 polypeptide consisting of SEQ ID NO:1.

In one embodiment, the antibody or antigen-binding fragment binds an HMGB B box or a biologically active fragment thereof. In another embodiment, the antibody or antigen-binding fragment binds an HMGB B box consisting of SEQ ID NO:5. In yet another embodiment, the antibody or antigen-binding fragment binds a biologically active fragment of an HMGB B box consisting of SEQ ID NO:45.

In one embodiment, the antibody or antigen-binding fragment binds an HMGB A box or a biologically active fragment thereof. In another embodiment, the antibody or antigen-binding fragment binds an HMGB A box consisting of SEQ ID NO:4. In yet another embodiment, the antibody or antigen-binding fragment binds a biologically active fragment of an HMGB A Box.

Other Inhibitors of HMGB Receptor Binding and/or HMGB Signaling

As described herein, the methods of the invention comprise administering an HMGB antagonist (e.g., an inhibitor of HMGB receptor binding and/or HMGB signaling). Such HMGB antagonists include, e.g., polypeptides comprising a high mobility group box (HMGB) A box or fragment thereof (as described herein), antibodies to HMGB, HMGB B boxes, HMGB A boxes and antigen-binding fragments thereof (as described herein), HMGB small molecule antagonists (e.g., ethyl pyruvate), antibodies to TLR2, soluble TLR2, TLR2 small molecule antagonists, TLR2 dominant mutant proteins, antibodies to TLR4, soluble TLR4, TLR4 small molecule antagonists, TLR4 dominant mutant proteins, antibodies to RAGE, soluble RAGE, RAGE small molecule antagonists (e.g., as taught in PCT Publication Nos. WO 01/99210, WO 02/069965 and WO 03/075921 and U.S. Published Application No. US 2002/0193432A1), and RAGE dominant mutant proteins. Inhibitors of HMGB receptor binding and/or signaling also include, e.g., antisense and small double-stranded interfering RNA (RNA interference (RNAi) that target HMGB, TLR2, TLR4 and/or RAGE proteins.

In one embodiment, the HMGB antagonist is an HMGB small molecule antagonist. As used herein, an HMGB small molecule antagonist is a molecule that antagonizes production of HMGB and/or one or more biological activities of HMGB (e.g., HMGB-mediated signaling, HMGB-mediated increase in inflammation, HMGB-mediated increase in release of a proinflammatory cytokine from a cell). Such HMGB small molecule antagonists include those small molecule antagonists that bind directly to HMGB, thereby inhibiting HMGB receptor binding and/or signaling, as well as those small molecule antagonists that do not bind to HMGB but antagonize production of HMGB and/or one or more biological activities of HMGB (e.g., HMGB-mediated signaling, HMGB-mediated increase in inflammation, HMGB-mediated increase in release of a proinflammatory cytokine from a cell). HMGB small molecule antagonists typically have a molecular weight of 1000 or less, 500 or less, 250 or less, or 100 or less. Suitable HMGB'small molecule antagonists include but are not limited to, an ester of an alpha-ketoalkanoic acid including, for example, ethyl pyruvate (see, e.g., PCT Publication WO 02/074301; the entire teachings of which are incorporated herein by reference).

For example, it has been shown that an ester of an alpha-ketoalkanoic acid can inhibit the release of protinflammatory cytokines, such as TNF, IL-1β and HMGB1 (see, e.g., PCT Publication WO 02/074301). Therefore, in one embodiment of the invention, the HMGB small molecule antagonist is an ester of an alpha-ketoalkanoic acid. In another embodiment, the HMGB small molecule antagonist is an ester of a C3 to C8, straight chained or branched alpha-ketoalkanoic acid. In an additional embodiment, the HMGB small molecule antagonist is selected from the group consisting of alpha-keto-butyrate, alpha-ketopentanoate, alpha-keto-3-methyl-butyrate, alpha-keto-4-methyl-pentanoate or alpha-keto-hexanoate. A variety of groups are suitable for the ester portion of the molecule, e.g., alkyl, aralkyl, alkoxyl, carboxyalkyl, glyceryl or dihydroxyacetone. Specific examples include ethyl, propyl, butyl, carboxymethyl, acetoxymethyl, carbethoxymethyl and ethoxymethyl. Ethyl esters are preferred. In a further embodiment, the HMGB small molecule antagonist is an ethyl, propyl, butyl, carboxymethyl, acetoxymethyl, carbethoxymethyl and ethoxymethyl ester. In an additional preferred embodiment, the HMGB small molecule antagonist is an ester of pyruvic acid. In a further preferred embodiment, the HMGB small molecule antagonist is ethyl pyruvate. Thiolesters (e.g., wherein the thiol portion is cysteine or homocysteine) are also included.

In another preferred embodiment, the HMGB small molecule antagonist is selected from the group consisting of ethyl pyruvate, propyl pyruvate, carboxymethyl pyruvate, acetoxymethyl pyruvate, carbethoxymethyl pyruvate, ethoxymethyl pyruvate, ethyl alpha-keto-butyrate, ethyl alpha-keto-pentanoate, ethyl alpha-keto-4-methyl-pentanoate and ethyl-keto-hexanoate.

It has been shown that HMGB polypeptides (e.g., HMGB1) bind Toll-like receptor 2 (TLR2) and that inhibition of the interaction between HMGB and TLR2 can decrease or prevent inflammation (U.S. Published Application No. 20040053841; the entire teachings of which are incorporated herein by reference). Therefore, in particular embodiments, the methods of the invention utilize agents that bind to HMGB and inhibit interaction between HMGB and TLR2 (e.g., antibodies to HMGB, antibodies to HMGB B boxes (as described herein), antibodies to HMGB A boxes, HMGB small molecule antagonists), as well as agents that bind to TLR2 and inhibit interaction between HMGB and TLR2 (e.g., antibodies to TLR2, TLR2 small molecule antagonists, soluble TLR2).

In one embodiment, the method comprises administering an HMGB antagonist that binds to TLR2 and inhibits interaction between HMGB and TLR2. Such HMGB antagonists include, e.g., an antibody or antigen-binding fragment that binds TLR2, a mutant of a natural ligand, a peptidomimetic, a competitive inhibitor of ligand binding. In one embodiment, the HMGB antagonist is a ligand that binds to TLR2 with greater affinity than HMGB binds to TLR2. Preferably the HMGB antagonist that binds to TLR2, thereby inhibiting binding by HMGB, does not significantly initiate or increase an inflammatory response, and/or does not significantly initiate or increase the release of a proinflammatory cytokine from a cell.

Examples of ligands that are known to bind TLR2 include heat shock protein 60, surfactant protein-A, monophosphoryl lipid A (Botler et al., Infect. Immun. 71(5): 2498-2507 (2003)), muramyl dipeptide (Beutler et al., Blood Cells Mol. Dis. 27(4):728-730 (2001)), yeast-particle zymosan, GPI anchor from Trypanosoma cruzi, Listeria monocytogenes, Bacillus, lipoteichoic acid, peptidoglycan, and lipopeptides from Streptococcus species, heat killed Mycobacteria tuberculosis, Mycobacteria avium lipopeptide, lipoarabinomannan, mannosylated phosphatidylinositol, Borrelia burgdorferi, Treponema pallidum, Treponema maltophilum (lipopeptides, glycolipids, outer surface protein A), and MALP-2 lipopeptides from Mycoplasma fermentans. Therefore, these molecules, as well as portions of these molecules that bind TLR2 can be used to inhibit the interaction between HMGB and TLR2 and can be used in the methods of the invention.

In another embodiment, the method comprises administering an HMGB antagonist that binds to HMGB, and prevents HMGB from binding to TLR2. Such an HMGB antagonist can be, for example, a soluble form of recombinant TLR2 (sTLR2) (i.e., TLR2 lacking the intracellular and transmembrane domains, as described, for example, by Iwaki et al., J. Biol. Chem. 277(27):24315-24320 (2002); the entire teachings of which are incorporated herein by reference), an anti-HMGB antibody or antigen-binding fragment (as described herein), or a non-HMGB antibody molecule (e.g., a protein, peptide, or small molecule antagonist) that binds HMGB and prevents it from binding to TLR2. The sTLR2 molecule can contain the extracellular domain (for example, amino acids 1-587 of the TLR2 amino acid sequence (e.g., GenBank Accession Number AAC34133). The sTLR molecule can also be modified with one of more amino acid substitutions and/or post-translational modifications, provided that such sTLR2 molecules have HMGB binding activity, which can be assessed using methods known in the art and/or described herein. Such sTLR2 molecules can be made, for example, using recombinant techniques. Preferably the sTLR2 has at least 70%, 75%, 80%, 85%, 90%, or 95%, identity to anmino acids 1-587 of GenBank Accession Number AAC34133. In another embodiment, the HMGB antagonist binds TLR2 at a site different than the HMGB binding site and blocks binding by HMGB (e.g., by causing a conformation change in the TLR2 protein or otherwise altering the binding site for HMGB). In another embodiment, the HMGB antagonist that is administered is a dominant negative mutant protein of TLR2.

It has also been shown that receptor signal transduction of HMGB1 occurs in part through Toll-like receptor 4 (TLR4) (Park, J. S. et al., J. Biol. Chem. 279(9):7370-77 (2004)). Therefore, in particular embodiments, the methods utilize HMGB antagonists that bind to TLR4 and inhibit HMBG1 binding and/or signaling and/or bind to HMGB and inhibit TLR4-mediated binding and/or signaling. Such HMGB antagonists include, e.g., antibodies to TLR4, TLR4 small molecule antagonists, soluble TLR4, dominant negative mutants of TLR4, mutants of a natural ligand of TLR4, peptidomimetics and competitive inhibitors of ligand binding to TLR4.

In one embodiment, the method comprises administering a soluble TLR4 polypeptide. It has been shown in mice that there is an alternatively spliced TLR4 mRNA (mTLR4), which expresses a partially secreted 20 kDa protein (soluble mTLR4; smTLR4) that inhibits LPS-mediated TNF-α production and NF-κB activation (Iwami, K-I et al., J. Immunol. 165:6682-6686 (2001); the entire teachings of which are incorporated herein by reference). In another embodiment, the HMGB antagonist that is administered is an antibody that binds TLR4 or an antigen-binding fragment thereof. Antibodies that bind TLR4 are known in the art (see, e.g., Tabeta, K. et al., Infect Immun. 68(6):3731-3735 (2000); and rabbit anti-TLR-4 (Catalog No. 36-3700; Zymed Laboratories, Inc., San Francisco, Calif.)).

It has been shown that HMGB polypeptides bind receptor for advanced glycation end-products (RAGE) and that receptor signal transduction occurs in part through RAGE (Andersson, U. et al., Scand. J. Infect. Dis. 35(9):577-84 (2003); Park, J. S. et al., J. Biol. Chem. 279(9):7370-77 (2004)). It has further been shown that inhibition of the interaction between HMGB and RAGE can decrease or prevent downstream signaling and cellular activation (Schmidt, A. M. et al., J. Clin. Invest. 108(7):949-955 (2001); Park, J. S. et al., J. Biol. Chem. 279(9):7370-77 (2004)). Therefore, in particular embodiments, the methods utilize HMGB antagonists that bind to HMGB and inhibit interaction between HMGB and RAGE (e.g., antibodies to HMGB, antibodies to HMGB B boxes (as described herein), antibodies to HMGB A boxes (as described herein), HMGB small molecule antagonists (as described herein)), as well as HMGB antagonists that bind to RAGE and inhibit interaction between HMGB and RAGE (e.g., antibodies to RAGE, RAGE small molecule antagonists (e.g., as taught in PCT Publication Nos. WO 01/99210, WO 02/069965 and WO 03/075921 and U.S. Published Application No. US 2002/0193432A1)), soluble RAGE (sRAGE; e.g., as taught in Schmidt, A. M. et al, J. Clin. Invest. 108(7):949-955 (2001), U.S. Application No. 2002/0122799 and PCT Publication No. WO 00/20621), RAGE dominant negative mutants (as taught in Schmidt, A. M. et at., J. Clin. Invest. 108(7):949-955 (2001)).

In one embodiment, the HMGB antagonist that is administered is an agent that binds to RAGE and inhibits interaction between HMGB and RAGE. Such HMGB antagonists include, e.g., an antibody or antigen-binding fragment that binds RAGE, a mutant of a natural ligand, a peptidomimetic and a competitive inhibitor of ligand binding. In one embodiment, the HMGB antagonist is a ligand that binds to RAGE with greater affinity than HMGB binds to RAGE. Preferably the HMGB antagonist that binds to RAGE, thereby inhibiting binding by HMGB, does not significantly initiate or increase an inflammatory response, and/or does not significantly initiate or increase the release of a proinflammatory cytokine from a cell.

Examples of ligands, other than HMBG1, that are known to bind RAGE include: AGEs (advanced glycation endproducts), S100/calgranulins and β-sheet fibrils (Schmidt, A. M. et at., J. Clin. Invest. 108(7):949-955 (2001)). Therefore, these molecules, as well as portions of these molecules that bind RAGE can be used to inhibit the interaction between HMGB and RAGE and can be used in the methods of the invention.

In another embodiment, the HMGB antagonist that is administered binds to HMGB and prevents HMGB from binding to RAGE. Such an HMGB antagonist can be, for example, a soluble truncated form of RAGE (sRAGE) (i.e., RAGE lacking its intracellular and transmembrane domains, as described, for example, by Schmidt, A. M. et al., J. Clin. Invest. 108(7):949-955 (2001), U.S. Application No. 2002/0122799 and PCT Publication No. WO 00/20621), an anti-HMGB antibody or antigen-binding fragment (as described herein), or a non-HMGB antibody molecule. (e.g., a protein, peptide, or non-peptidic small molecule) that binds HMGB and prevents it from binding to RAGE. The sRAGE molecule can be modified with one of more amino acid substitutions and/or post-translational modifications provided such sRAGE molecules have HMGB binding activity, which can be assessed using methods known in the art. Such sRAGE molecules can be made, for example, using recombinant techniques. In another embodiment, the HMGB antagonist binds RAGE at a site different than the HMGB binding site and blocks binding by HMGB (e.g., by causing a conformation change in the RAGE protein or otherwise altering the binding site for HMGB). In another embodiment, the HMGB antagonist is a dominant negative mutant protein of RAGE. Dominant negative mutant RAGE proteins, which are capable of binding to RAGE but suppress RAGE-mediated signaling are known in the art (see e.g., Schmidt, A. M. et al., J. Clin. Invest. 108(7):949-955 (2001)).

In a particular embodiment, the HMGB antagonist is not an anti-TLR2 antibody or antigen-binding fragment thereof. In another embodiment, the HMGB antagonist is not an antibody that binds HMGB1 (an anti-HMGB1 antibody) or an antigen-binding fragment thereof. In yet another embodiment, the HMGB antagonist is not an antibody that binds HMGB (an anti-HMGB antibody) or an antigen-binding fragment thereof. In another embodiment, the HMGB antagonist is not soluble RAGE (i.e., a portion of the RAGE receptor that binds HMGB1). In another embodiment, the HMGB antagonist is non-microbial (i.e., is not a microbe, derived from a microbe, or secreted or released from a microbe). In still another embodiment, the HMGB antagonist is a mammalian HMGB antagonist (i.e., is a molecule that naturally exists in a mammal, is derived from a molecule that naturally exists in a mammal, or is secreted or released from a mammalian cell), for example, a human HMGB antagonist.

In a particular embodiment, the HMGB antagonist is a small molecule (i.e., having a molecular weight of 1000 or less, 500 or less, 250 or less or 100 or less). In another embodiment the HMGB antagonist is a short peptide, having, for example, 50 or fewer amino acids, 30 or fewer amino acids, 25 or fewer amino acids, 20 or fewer amino acids, 10 or fewer amino acids, or 5 or fewer amino acids.

As described herein, HMGB antagonists include, e.g., antisense nucleic acids and small double-stranded interfering RNA (RNA interference (RNAi)) that target HMGB, TLR2, TLR4 and/or RAGE. Antisense nucleic acids and RNAi can be used to decrease expression of a target molecule, e.g., HMGB, TLR2, TLR4, RAGE, as is known in the art.

Production and delivery of antisense nucleic acids and RNAi is known in the art (e.g., as taught in PCT Publication WO 2004/016229). In one embodiment, small double-stranded interfering RNA (RNA interference (RNAi)) can be used (e.g., RNAi that targets HMGB, TLR2, TLR4 and/or RAGE) in the methods of the invention. RNAi is a post-transcription process, in which double-stranded RNA is introduced, and sequence-specific gene silencing results, though catalytic degradation of the targeted mRNA (see, e.g., Elbashir, S. M. et al., Nature 411:494-498 (2001); Lee, N. S., Nature Biotech. 19:500-505 (2002); and Lee, S-K. et al., Nature Medicine 8(7):681-686 (2002); the entire teachings of these references are incorporated herein by reference.

RNAi is used routinely to investigate gene function in a high throughput fashion or to modulate gene expression in human diseases (Chi et al., Proc. Natl. Acad Sci. U.S.A., 100(1):6343-6346 (2003)). Introduction of long double stranded RNA leads to sequence-specific degradation of homologous gene transcripts. The long double stranded RNA is metabolized to small 21-23 nucleotide siRNA (small interfering RNA). The siRNA then binds to protein complex RISC (RNA-induced silencing complex) with dual function helicase. The helicase has RNase activity and is able to unwind the RNA. The unwound siRNA allows an antisense strand to bind to a target. This results in sequence dependent degradation of cognate mRNA. Aside from endogenous RNAi, exogenous RNAi, chemically synthesized or recombinantly produced RNAi can also be used in the compositions and methods of the invention.

In one embodiment, the methods of the invention utilize aptamers of HMGB (e.g., aptamers of HMGB1). As is known in the art, aptamers are macromolecules composed of nucleic acid (e.g., RNA, DNA) that bind tightly to a specific molecular target (e.g., an HMGB protein, an HMGB box (e.g., an HMGB A box, an HMGB B box), an HMGB polypeptide and/or an HMGB epitope). A particular aptamer may be described by a linear nucleotide sequence and is typically about 15-60.nucleotides in length. The chain of nucleotides in an aptamer form intramolecular interactions that fold the molecule into a complex three-dimensional shape, and this three-dimensional shape allows the aptamer to bind tightly to the surface of its target molecule. Given the extraordinary diversity of molecular shapes that exist within the universe of all possible nucleotide sequences, aptamers may be obtained for a wide array of molecular targets, including proteins and small molecules. In addition to high specificity, aptamers have very high affinities for their targets (e.g., affinities in the picomolar to low nanomolar range for proteins). Aptamers are chemically stable and can be boiled or frozen without loss of activity. Because they are synthetic molecules, they are amenable to a variety of modifications, which can optimize their function for particular applications. For example, aptamers can be modified to dramatically reduce their sensitivity to degradation by enzymes in the blood for use in in vivo applications. In addition, aptamers can be modified to alter their biodistribution or plasma residence time.

Selection of aptamers that can bind HMGB or a fragment thereof (e.g., HMGB1 or a fragment thereof) can be achieved through methods known in the art. For example, aptamers can be selected using the SELEX (Systematic Evolution of Ligands by Exponential Enrichment) method (Tuerk, C., and Gold, L., Science 249:505-510 (1990)). In the SELEX method, a large library of nucleic acid molecules (e.g., 10¹⁵ different molecules) is produced and/or screened with the target molecule (e.g., an HMGB protein, an HMGB box (e.g., an HMGB A box, an HMGB B box), an HMGB polypeptide and/or an HMGB epitope). The target molecule is allowed to incubate with the library of nucleotide sequences for a period of time. Several methods, known in the art, can then be used to physically isolate the aptamer target molecules from the unbound molecules in the mixture, which can be discarded. The aptamers with the highest affinity for the target molecule can then be purified away from the target molecule and amplified enzymatically to produce a new library of molecules that is substantially enriched for aptamers that can bind the target molecule. The enriched library can then be used to initiate a new cycle of selection, partitioning, and amplification. After 5-15 cycles of this iterative selection, partitioning and amplification process, the library is reduced to a small number of aptamers that bind tightly to the target molecule. Individual molecules in the mixture can then be isolated, their nucleotide sequences determined, and their properties with respect to binding affinity and specificity measured and compared. Isolated aptamers can then be further refined to eliminate any nucleotides that do not contribute to target binding and/or aptamer structure, thereby producing aptamers truncated to their core binding domain. See Jayasena, S. D. Clin. Chem. 45:1628-1650 (1999) for review of aptamer technology; the entire teachings of which are incorporated herein by reference).

In particular embodiments, the methods of the invention utilize aptamers having the same or similar binding specificity as described herein for HMGB antagonists (e.g., binding specificity for an HMGB polypeptide, fragment of an HMGB polypeptide (e.g., an HMGB A box, an HMGB B box), epitopic region of an HMGB polypeptide). In particular embodiments, the aptamers of the invention can bind to an HMGB polypeptide or fragment thereof and inhibit one or more functions of the HMGB polypeptide. As described herein, functions of HMGB polypeptides include, e.g., increasing inflammation, increasing release of a proinflammatory cytokine from a cell, binding to RAGE, binding to TLR2, chemoattraction. In a particular embodiment, the aptamer binds HMGB1 (e.g., human HMGB1) or a fragment thereof (e.g., an A box, a B box) and inhibits one or more functions of the HMGB polypeptide (e.g., inhibits release of a proinflammatory cytokine from a vertebrate cell treated with HMGB).

### Methods of Treatment

In one embodiment, the invention is a method of treating an inflammatory skin condition in a subject comprising administering to said subject an HMGB antagonist. As described herein, HMGB antagonists inhibit proinflammatory cytokine release and inflammatory cytokine cascades, and can be used to treat inflammatory skin conditions. Further, as demonstrated herein, in addition to being actively secreted by macrophages/monocytes in response to inflammatory stimuli (Wang H., et al., Science 285:248-51 (1999)), HMGB1 is secreted by keratinocytes (see, e.g., Example 3).

As used herein, a “cytokine” is a soluble protein or peptide that is naturally produced by mammalian cells and that regulates immune responses and mediates cell-cell interactions. Cytokines can, either under normal or pathological conditions, modulate the functional activities of individual cells and tissues. A proinflammatory cytokine is a cytokine that is capable of causing one or more of the following physiological reactions associated with inflammation or inflammatory conditions: vasodilation, hyperemia, increased permeability of vessels with associated edema, accumulation of granulocytes and mononuclear phagocytes, and deposition of fibrin. In some cases, the proinflammatory cytokine can also cause apoptosis, such as in chronic heart failure, where TNF has been shown to stimulate cardiomyocyte apoptosis (Pulkki, Ann. Med. 29:339-343 (1997); and Tsutsui et al., Immunol. Rev. 174:192-209 (2000)).

Nonlimiting examples of proinflammatory cytokines are tumor necrosis factor (TNF), interleukin (IL)-1α, IL-1β, IL-6, IL-8, IL-18, interferon-γ, HMG-1, and macrophage migration inhibitory factor (MIF). In a particular embodiments, the proinflammatory cytokine is TNF (e.g., TNF-α)). Proinflammatory cytokines are to be distinguished from anti-inflammatory cytokines, such as IL-4, IL-10, and IL-13, which are not mediators of inflammation.

In many instances, proinflammatory cytokines are produced in an inflammatory cytokine cascade, defined herein as an in vivo release of at least one proinflammatory cytokine in a mammal, wherein the cytokine release, directly or indirectly (e.g., through activation of, production of, or release of, one or more cytokines or other molecules involved in inflammation from a cell), stimulates a physiological condition of the mammal. Thus, an inflammatory cytokine cascade is inhibited in embodiments of the invention where proinflammatory cytokine release causes a deleterious physiological condition.

Inhibition of release of a proinflammatory cytokine from a cell can be measured according to methods known to one skilled in the art. For example, TNF release from a cell can be measured using a standard murine fibroblast L929 (ATCC, American Type Culture Collection, Rockville, Md.) cytotoxicity bioassay (Bianchi et al., J. Exp. Med. 183:927-936 (1996)) with the minimum detectable concentration of 30 pg/ml. The L929 cytotoxicity bioassay is carried out as follows. RAW 264.7 cells are cultured in RPMI 1640 medium (Life Technologies, Grand Island, N.Y.) supplemented with 10% fetal bovine serum (Gemini, Catabasas, Calif.), penicillin and streptomycin (Life Technologies). Polymyxin (Sigma, St. Louis, Mo.) is added at 100 units/ml to suppress the activity of any contaminating LPS. Cells are incubated with an agent (e.g., an HMGB antagonist as described herein) in Opti-MEM I medium for 8 hours, and conditioned supernatants (containing TNF which has been released from the cells) are collected. TNF which has been released from the cells is measured using a standard murine fibroblast L929 (ATCC) cytotoxicity bioassay (Bianchi et al., supra) with the minimum detectable concentration of 30 pg/ml. Recombinant mouse TNF is obtained from R&D Systems Inc. (Minneapolis, Minn.) and is used as a control in these experiments. Methods for measuring release of other cytokines from cells are known in the art.

### Inflammatory Skin Conditions

Inflammatory cytokine cascades contribute to deleterious characteristics, including inflammatory conditions and cellular apoptosis. As described herein, in one embodiment, the invention is a method of treating an inflammatory skin condition comprising administering to a subject an HMGB antagonist. Inflammatory skin conditions that can be treated by the methods of the invention are well known in the art and include, e.g., acne, rosacea, psoriasis, dermatitis (including atopic, contact, seborrheic, nummular, exfoliative, periorial and stasis dermatitis), dermatitis herpetiformis, allergic skin reactions, cold sores, dry skin, allergic skin conditions, insect bites, burns, pruritis, urticaria, erythematosus multiforme, erythema toxicum, folliculitis, impetigo, cutaneous lupus erythematosus (including acute CLE, subacute CLE, chronic CLE and discoid lupus erythematosus), cellulitis, acute lymphangitis, lymphadenitis, erysipelas, cutaneous abcesses, necrotizing subcutaneous infections, staphylococcal scalded skin syndrome, folliculitis, furuncles, hidradenitis suppurativa, carbuncles, paronychial infections, erythasma, pemphigus vulgaris, pemphigus foliaceus, paraneoplastic pemphigus, bullous pemphigoid, pemphigoid gestationis, linear IgA disease, epidermolysis bullosa acquisita, cicatrical pemphigoid, scleroderma and morphea (localized scleroderma) and photosensitivity diseases (e.g., polymorphous light eruption, photoallergy).

In one embodiment, the invention is a method of treating an inflammatory skin condition selected from the group consisting of psoriasis, acne, pruritis, rosacea, erythematosus multiforme, erythema toxicum, folliculitis, impetigo, cutaneous lupus erythematosus (CLE), cold sores, dry skin, allergic skin conditions and insect bites.

In another embodiment, the invention is a method of treating dermatitis (e.g., atopic dermatitis, contact dermatitis, seborrheic dermatitis, nummular dermatitis, exfoliative dermatitis, periorial dermatitis and stasis dermatitis). In yet another embodiment, the invention is a method of treating atopic dermatitis, seborrheic dermatitis, nummular dermatitis, exfoliative dermatitis, periorial dermatitis and stasis dermatitis.

In a particular embodiment, the skin condition to be treated is not eczema, dermatitis, allergic contact dermatitis, psoriasis, alopecia, burns, dermatomyositis, sunburn, urticaria warts or wheals.

In one embodiment, the invention is a method of treating cutaneous lupus erythematosus (CLE) (e.g., acute cutaneous lupus erythematosus (ACLE), subacute cutaneous lupus erythematosus (SCLE), chronic cutaneous lupus erythematosus (CCLE) (e.g., discoid lupus erythematosus (DLE))) in a subject comprising administering an HMGB1 antagonist. As described and exemplified herein, HMGB1 expression was increased in the lesions of patients with cutaneous lupus (Example 1).

Moreover, as exemplified herein, HMGB1 plays a role in the development and progression of Erythema toxicum and is secreted by keratinocytes in response to the first colonization of the skin by microorganisms (e.g., bacteria) in human newborns (Example 3). Therefore in one embodiment, the invention is a method of treating a bacterially-mediated inflammatory skin condition. Such bacterially-mediated inflammatory skin conditions (many of which are treated using antibiotic compounds) are known in the art and include, e.g., acne, rosacea, cellulitis, acute lymphangitis, lymphadenitis, erysipelas, cutaneous abcesses, necrotizing subcutaneous infections, staphylococcal scalded skin syndrome, folliculitis, furuncles, hidradenitis suppurativa, carbuncles, paronychial infections and erythasma, nummular dermatitis, perioral dermatitis. In one embodiment, the method further comprises administering an antibiotic compound with the HMGB antagonist (either prior to, concurrently, or after administration of the HMGB antagonist).

In a particular embodiment, the invention is a method of treating a bacterially-mediated inflammatory skin condition selected from the group consisting of acne and rosacea. As exemplified herein, HMGB1 is secreted from keratinocytes in response to microbial invasion, and therefore, inflammatory skin conditions mediated by bacteria (e.g., acne, rosacea) can be treated using the HMGB antagonists described herein.

In one embodiment, the invention is a method of treating erythema toxicum comprising administering an HMGB1 antagonist. As exemplified herein, HMGB1 was secreted from keratinocytes in inflammatory lesions of patients with erythema toxicum (Example 2).

As demonstrated herein, keratinocytes can secrete HMBG1, and in the keratinocytes of inflammatory lesions, HMGB1 is found in the cytoplasm and extracellular space. Thus, in one embodiment, the invention is a method of inhibiting release of HMGB1 from keratinocytes comprising administering an HMBG1 antagonist. Keratinocytes play an important role in host defense (Wang H., et al., Surgery 126:389-392 (1999)) and therefore, the administration of an HMGB antagonist can be of benefit to inflammatory skin conditions involving keratinocyte proinflammatory cytokine release.

In addition, as is known in the art, keratinocytes control melanocyte growth and behavior through a complex system of paracrine growth factors and cell-cell adhesion molecules (Haass, N. K., et al., Pigment Cell Res. 18(3):150-159 (2005)). Alteration of this delicate homeostatic balance and can lead to altered expression of cell-cell adhesion and cell communication molecules and to the development of melanoma. Id. Inflammation is known to play an important role in cancer (e.g., melanoma). For example, Waterston et al. teach that immunization of mice with a TNF autovaccine produced a 100-fold antibody response to TNF, as compared to immunization with a phosphate-buffered saline vehicle control, and significantly reduces both the number and size of metastases of B16F10 melanoma cells (Waterston, A. M. et al., Br. J. Cancer, 90(6):1279-84 (2004)). Therefore, in one embodiment, the invention is a method of treating melanoma comprising administering to a subject an HMGB antagonist of the invention.

As exemplified and described herein, the expression of HMBG1 is increased in cutaneous lesions of lupus erythematosus (CLE). Cutaneous manifestations of lupus erythematosus can be divided into acute (ACLE), subacute (SCLE), chronic (CCLE) lupus erythematosus (e.g., discoid lupus erythematosus (DLE)). In addition to cutaneous manifestations of lupus erythematosus, the condition can occur in other forms. For example, other forms of lupus erythematosus include systemic lupus erythematosus, drug-induced lupus erythematosus and neonatal lupus erythematosus. Accordingly, in one embodiment, the invention is a method of treating lupus erythematosus (LE) comprising administering an HMGB antagonist of the invention. In another embodiment, the invention is a method of treating one or more forms of lupus erythematosus (e.g., cutaneous lesions of lupus erythematosus (CLE), systemic lupus erythematosus, drug-induced lupus erythematosus, neonatal lupus erythematosus) comprising administering an HMGB antagonist of the invention.

As exemplified herein, HMGB1 is expressed in healthy and UVB-irradiated skin of CLE patients, as well as healthy control subjects (Example 2). However, in CLE subjects, exposure to UV rays altered the expression of HMBG1, such that it mirrored the appearance of clinical symptoms. Accordingly, in one embodiment, the invention is a method of preventing or decreasing tissue damage (e.g., skin damage) from exposure to UV comprising administering an HMGB antagonist of the invention.

In the methods of the invention, administration of an HMGB antagonist inhibits or decreases the release of proinflammatory cytokines. As used herein, the terms “inhibit” or “decrease” encompasses at least a small but measurable reduction in proinflammatory cytokine release. In preferred embodiments, the release of the proinflammatory cytokine is inhibited by at least 10%, 20%, 25%, 30%, 40%, 50%, 75%, 80%, or 90%, over non-treated controls. Inhibition can be assessed using methods described herein and/or other methods known in the art. Such reductions in proinflammatory cytokine release are capable of reducing the deleterious effects of an inflammatory cytokine cascade involved in an inflammatory skin condition.

The present invention provides a method of treating an inflammatory skin condition in a subject comprising administering to said subject an HMGB antagonist. In one embodiment, the invention is a method of treating an inflammatory skin condition in a subject at risk for having an inflammatory skin condition. In the methods, an effective amount of an HMGB antagonist is administered. As used herein, an “effective amount” is an amount sufficient to prevent or decrease an inflammatory response, and/or to ameliorate and/or decrease the longevity of symptoms associated with an inflammatory response. Methods for determining whether an HMGB antagonist inhibits an inflammatory condition are known to one skilled in the art and/or are described herein. Inhibition of the release of a proinflammatory cytokine from a cell can be measured by any method known to one of skill in the art, for example, using the L929 cytotoxicity assay described herein. The inflammatory skin condition to be treated can be one in which the inflammatory cytokine cascade is activated.

Preferably, the HMGB antagonist is administered to a subject in need thereof in an amount sufficient to inhibit release of proinflammatory cytokine from a cell and/or to treat an inflammatory condition. In one embodiment, release of the proinflammatory cytokine is inhibited by at least 10%, 20%, 25%, 50%, 75%, 80%, 90%, or 95%, as assessed using methods described herein and/or other methods known in the art.

The terms “therapy,” “therapeutic,” and “treatment” as used herein, refer to ameliorating symptoms associated with a disease or condition, for example, an inflammatory skin disease or an inflammatory skin condition, including preventing or delaying the onset of the disease symptoms, and/or lessening the severity or frequency of symptoms of the disease or condition. The terms “subject” and “individual” are defined herein to include animals such as mammals, including, but not limited to, primates, cows, sheep, goats, horses, dogs, cats, rabbits, guinea pigs, rats, mice or other bovine, ovine, equine, canine, feline, rodent, or murine species. In a preferred embodiment, the animal is a human.

The HMGB antagonists used in the methods of the invention can optionally include a carrier (e.g., a pharmaceutically acceptable carrier). The carrier included with the HMGB antagonist is chosen based on the expected route of administration of the HMGB antagonist in therapeutic applications. The route of administration of the HMGB antagonist depends on the condition to be treated. The dosage of the HMGB antagonist to be administered can be determined by the skilled artisan without undue experimentation in conjunction with standard dose-response studies. Relevant circumstances to be considered in making those determinations include the condition or conditions to be treated, the choice of HMGB antagonist to be administered, the age, weight, and response of the individual patient, and the severity of the patient's symptoms. Typically, an effective amount can range from 0.01 mg per day to about 100 mg per day for an adult. Preferably, the dosage ranges from about 1 mg per day to about 100 mg per day or from about 1 mg per day to about 10 mg per day. Depending on the condition, the combination therapy composition can be administered orally, parenterally, intranasally, vaginally, rectally, lingually, sublingually, buccally, intrabuccally and/or transdermally to the patient.

Accordingly, HMGB antagonist compositions designed for oral, lingual, sublingual, buccal and intrabuccal administration can be made without undue experimentation by means well known in the art, for example, with an inert diluent or with an edible carrier. The HMGB antagonist composition may be enclosed in gelatin capsules or compressed into tablets. For the purpose of oral therapeutic administration, the HMGB antagonist compositions of the present invention may be incorporated with excipients and used in the form of tablets, troches, capsules, elixirs, suspensions, syrups, wafers, chewing gums, and the like.

Tablets, pills, capsules, troches and the like may also contain binders, recipients, disintegrating agent, lubricants, sweetening agents, and/or flavoring agents. Some examples of binders include microcrystalline cellulose, gum tragacanth and gelatin. Examples of excipients include starch and lactose. Some examples of disintegrating agents include alginic acid, corn starch, and the like. Examples of lubricants include magnesium stearate and potassium stearate. An example of a glidant is colloidal silicon dioxide. Some examples of sweetening agents include sucrose, saccharin, and the like. Examples of flavoring agents include peppermint, methyl salicylate, orange flavoring, and the like. Materials used in preparing these various compositions should be pharmaceutically pure and non-toxic in the amounts used.

The HMGB antagonists of the present invention can be administered parenterally, such as, for example, by intravenous, intramuscular, intrathecal and/or subcutaneous injection. Parenteral administration can be accomplished by incorporating the HMGB antagonist into a solution or suspension. Such solutions or suspensions may also include sterile diluents, such as water for injection, saline solution, fixed oils, polyethylene glycols, glycerine, propylene glycol and/or other synthetic solvents. Parenteral formulations may also include antibacterial agents, for example, benzyl alcohol and/or methyl parabens; antioxidants, for example, ascorbic acid and/or sodium bisulfite; and chelating agents, for example, EDTA. Buffers, such as-acetates, citrates and phosphates, and agents for the adjustment of tonicity, such as sodium chloride and dextrose, may also be added. The parenteral preparation can be enclosed in ampules, disposable syringes and/or multiple dose vials made of glass or plastic.

Rectal administration includes administering the HMGB antagonist into the rectum and/or large intestine. This can be accomplished using suppositories and/or enemas. Suppository formulations can be made by methods known in the art. For example, suppository formulations can be prepared by heating glycerin to about 120° C., dissolving the HMGB antagonist in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves, and the like. For many inflammatory skin conditions, transdermal administration is the preferred mode of administration.

HMGB antagonists can be administered nasally to a patient. As used herein, nasally administering or nasal administration includes administering the HMGB antagonists to the mucous membranes of the nasal passage and/or nasal cavity of the patient. Pharmaceutical compositions for nasal administration of an HMGB antagonist include therapeutically effective amounts of the HMGB antagonist prepared by well-known methods to be administered, for example, as a nasal spray, nasal drop, suspension, gel, ointment, cream and/or powder. Administration of the composition may also take place using a nasal tampon and/or nasal sponge.

If desired, the HMGB antagonist compositions described herein can also include one or more additional agents used to treat an inflammatory condition. Such agents are known to one of skill in the art. The agent may be, for example, an antagonist of an early sepsis mediator. As used herein, an early sepsis mediator is a proinflammatory cytokine that is released from cells soon (i.e., within 30-60 min.) after induction of an inflammatory cytokine cascade (e.g., exposure to LPS). Nonlimiting examples of these cytokines are IL-1α, IL-1β, IL-6, PAF, and MIF. Also included as early sepsis mediators are receptors for these cytokines (for example, tumor necrosis factor receptor type 1) and enzymes required for production of these cytokines (for example, interleukin-1β converting enzyme). Antagonists of any early sepsis mediator, now known or later discovered, can be useful for these embodiments by further inhibiting an inflammatory cytokine cascade.

Nonlimiting examples of antagonists of early sepsis mediators are antisense compounds that bind to the mRNA of the early sepsis mediator, preventing its expression (see, e.g., Ojwang et al., Biochemistry 36:6033-6045, 1997; Pampfer et al., Biol. Reprod. 52:1316-1326, 1995; U.S. Pat. No. 6,228,642; Yahata et al., Antisense Nucleic Acid Drug Dev. 6:55-61, 1996; and Taylor et al., Antisense Nucleic Acid Drug Dev. 8:199-205, 1998), ribozymes that specifically cleave the mRNA of the early sepsis mediator (see, e.g., Leavitt et al., Antisense Nucleic Acid Drug Dev. 10:409-414, 2000; Kisich et al., 1999; and Hendrix et al., Biochem. J. 314 (Pt. 2):655-661, 1996), and antibodies that bind to the early sepsis mediator and inhibit their action (see, e.g., Kam and Targan, Expert Opin. Pharmacother. 1:615-622, 2000; Nagahira et al., J. Immunol. Methods 222:83-92, 1999; Lavine et al., J. Cereb. Blood Flow Metab. 18:52-58, 1998; and Holmes et al., Hybridoma 19:363-367, 2000). An antagonist of an early sepsis mediator, now known or later discovered, is envisioned as within the scope of the invention. The skilled artisan can determine the amount of early sepsis mediator to use in these compositions for inhibiting any particular inflammatory cytokine cascade without undue experimentation, e.g., using routine dose-response studies.

Other agents that can be administered with the HMGB antagonists described herein include, e.g., Vitaxin™ and other antibodies targeting αaβ3 integrin (see, e.g., U.S. Pat. No. 5,753,230, PCT Publication Nos. WO 00/78815 and WO 02/070007; the entire teachings of all of which are incorporated herein by reference) and anti-IL-9 antibodies (see, e.g., PCT Publication No. WO 97/08321; the entire teachings of which are incorporated herein by reference).

In one embodiment, the HMGB antagonists of the invention are administered with inhibitors of TNF biological activity. Such inhibitors of TNF activity include, e.g., peptides, proteins, synthesized molecules, for example, synthetic organic molecules, naturally-occurring molecule, for example, naturally occurring organic molecules, nucleic acid molecules, and components thereof Preferred examples of agents that inhibit TNF biological activity include infliximab (Remicade; Centocor, Inc., Malvern, Pa.), etanercept (Immunex; Seattle, Wash.), adalimuniab (D2E7; Abbot Laboratories, Abbot Park Ill.), CDP870 (Pharmacia Corporation; Bridgewater, N.J.) CDP571 (Celltech Group plc, United Kingdom), Lenercept (Roche, Switzerland), and Thalidomide.

In another embodiment, an HMGB antagonist is administered with an inhibitor of complement biological activity. As used herein, “an inhibitor of complement biological activity” or “an agent that inhibits complement biological activity” is an agent that decreases one or more of the biological activities of the complement system. Examples of complement biological activity include, but are not limited to, cell lysis, development of an inflammatory response, opsonization of antigen, viral neutralization, and clearance of immune complexes. Components of the complement system participate in the development of an inflammatory response by degranulating mast cells, basophils, and eosinophils, aggregation of platelets, and release of neutrophils from bone marrow. Agents that inhibit complement biological activity include, e.g., agents that inhibit (decrease) the interaction between a complement component and its receptor(s), agents that inhibit (decrease) formation of the MAC, agents that inhibit a key protein in the complement cascade, agents that inhibit conversion of complement C5 to C5a and C5b, and agents that inhibit the action of complement-derived anaphalytoxins C3a and C5a. Such agents include, but are not limited to peptides, proteins, synthesized molecules (for example, synthetic organic molecules), naturally-occurring molecule (for example, naturally occurring organic molecules), nucleic acid molecules, and components thereof. Preferred examples of agents that inhibit complement biological activity include agents that inhibit expression or activity or one or more of the following components of the complement system: C1q, C1r, C1s, Factor D, Factor B, Properdin, C2, C3, C4, C5, C6, C7, C8, C9, C3 convertase, C5 convertase, as well as fragments of components that are produced upon activation of complement, for example, fragment 2a, 2b, 3a, 3b, 4a, 4b, 5a, and/or 5b.

Examples of agents that inhibit complement biological activity include, but are not limited to: C5 inhibitors, for example, 5G1.1 (also known as Eculizumab; Alexion Pharmaceuticals, Inc., Cheshire, Conn.) and h5G1.1-SC (also known as Pexelizumab, Alexion Pharmaceuticals Inc., Cheshire, Conn.); C5a receptor antagonists, for example, NGD 2000-1 (Neurogen, Corp., Branford, Conn.) and AcPhe[Om-Pro-D-Cyclohexylalanine-Trp-Arg] (AcF-[OPdChaWR]; see, e.g., Strachan, A. J. et al., Br. J. Pharmacol. 134(8):1778-1786 (2001)); C1 esterase inhibitor (C1-IH); Factor H (inactive C3b); Factor I (inactive C4b); soluble complement receptor type 1 (sCR1; see, e.g., U.S. Pat. No. 5,856,297) and sCR1-sLe(X) (see, e.g., U.S. Pat. No. 5,856,300; membrane cofactor protein (MCP), decay accelerating factor (DAF) and CD59 and soluble recombinant forms thereof (Ashgar, S. S. et al., Front Biosci. 5:E63-E81 (2000) and Sohn, J. H. et al., Invest. Opthamol. Vis. Sci. 41(13):4195-4202 (2000)); Compstatin (Morikis et al., Protein Sci. 7:619-627 (1998); Sahu, A. et al., J. Immunol. 165:2491-2499 (2000)); chimeric complement inhibitor proteins having at least two complementary inhibitory domains (see, e.g., U.S. Pat. Nos. 5,679,546, 5,851,528 and 5,627,264); and small molecule antagonists (see, e.g., PCT Publication No. WO 02/49993, U.S. Pat. Nos. 5,656,659, 5,652,237, 4,510,158, 4,599,203 and 4,231,958). Other known complement inhibitors are known in the art and are encompassed by the invention. In addition, methods for measuring complement activity (e.g., to identify agents that inhibit complement activity) are known in the art. Such methods include, e.g., using a 50% hemolytic complement (CH₅₀) assay (see, e.g., Kabat et al., Experimental Immunochemistry, 2nd Ed. (Charles C. Thomas, Publisher, Springfield, Ill.), p. 133-239 (1961)), using an enzyme immunoassay (EIA), using a liposome immunoassay (LIA) (see, e.g., Jaskowski et al., Clin. Diagn. Lab. Immunol 6(1):137-139 (1999)).

In a particular embodiment, the method further comprises administering one or more other cosmetic and/or pharmaceutical agents, which are known in the art for treating adverse skin conditions or cosmetic skin conditions. Cosmetic and pharmaceutical agents include, e.g., chemical substances (natural or synthetic) that are intended for application (e.g., topical application) to the skin or its appendages in human and animals. Some examples of cosmetic and pharmaceutical agents include age spot- and keratoses-removing agents, analgesics, anesthetics, antiacne agents antibacterial agents, antiyeast agents, antifungal agents, antiviral agents, antiburn agents, antidandruff agents, antidermatitis agents, antipruritic agents, antiperspirants, antiinflammatory agents, antihyperkeratolytic agents, ant-dry skin agents, antipsoriatic agents, antiseborrheic agents, astringents, softeners, emollient agents, coal tar, bath oils, sulfur, rinse conditioners, foot care agents, hair growth agents, powder, shampoos, skin bleaches, skin protectants, soaps, cleansers, antiaging agents, sunscreen agents, wart removers, wet dressings, vitamins, tanning agents, topical antihistamine agents, hormones, vasodilators, retinoids, and other dermatological agents. Such cosmetic and/or pharmaceutical agents typically would be administered in a therapeutically or cosmetically effective amount, as determined as appropriate by a clinician, or other health care or cosmetic care professional.

### Exemplification

### Example 1

### Increased Expression and Cytoplasmic/Extracellular Localization of the Pro-Inflammatory Cytokine HMGB1 in Cutaneous Lesions of Lupus Erythentatosus

### Introduction

We investigated the role of HMBG1 in cutaneous manifestations of lupus, by monitoring the expression and subcellular localization of HMGB1 and the pro-inflammatory cytokines, TNF-α and IL-1β, in punch biopsies from patients with cutaneous lupus erythematosus (CLE). Specifically, HMGB1 expression and localization was analyzed in lesions from patients with subacute cutaneous lupus erythematosus (SCLE) and discoid lupus erythematosus (DLE). SCLE is defined as a non-scarring skin eruption that is associated with Ro/SSA-autoantibodies and photosensitivity. Discoid lupus erythematosus (DLE) is characterized by skin lesions consisting of red plaques with thick scale and follicular plugs. We also performed single nucleotide polymorphism (SNP) analysis on samples from these CLE patients to determine the frequency of a particular TNF-α promoter polymorphism (e.g., the'308 TNF polymorphism), which has been associated with SCLE and increased TNF production (Werth, V. P., et al., J. Invest. Dermatol. 115 (4):726-730 (2000)).

### Materials and Methods

### Patient Samples and SNP Analysis

To investigate the role of HMGB1 in the pathogenesis of cutaneous lupus erythematosus (CLE), skin punch biopsies were obtained from ten patients (seven females and three males) with CLE, who were selected for the study on the basis of having spontaneous active skin lesions during clinical examination. In this study the diagnosis of CLE was based on clinical and histopathologic findings. Of the ten patients with CLE, six had subacute cutaneous lupus erythematosus (SCLE), and 4 had discoid lupus erythematosus (DLE). Seven of these patients also had systemic manifestations of lupus. Skin biopsies from three healthy female volunteers served as normal control biopsies. For the study, 3 mm punch biopsy samples were obtained from the active zone of the lesion and from unaffected buttock skin of each CLE patient. A 3 mm punch biopsy from buttock skin was obtained from each healthy control patient as well. DNA was extracted from peripheral blood mononuclear cells of the ten CLE patients and was subsequently analyzed for TNF-α promoter single nucleotide polymorphisms (SNPs), according to a previously-described procedure (Padyukov, L., et al., Genes Immun. 2:280-283 (2001)). This study was supported by the Human Ethics Committee Region North and informed consent was provided by all of the patients.

### Immunohistochemical Staining

3 mm punch biopsy samples were snap-frozen on dry ice, and all samples were stored at −70° C., until sectioned in a cryostat. The sections, which were 7 μm thick, were placed on chrome gelatin-coated slides. The slides with the sections were air dried for 30 minutes before fixation in 2% formaldehyde, which was diluted in phosphate buffered saline (PBS). Subsequently, the slides were rinsed in PBS and kept at −70° C. until used. To block endogenous peroxidase activity, the slides were washed in PBS-Saponin for 10 minutes, followed by a 60 minute incubation in 1% H₂O₂, 2% NaN₃, 0.1% Saponin in PBS in the dark. Following this incubation, the slides were washed three times in PBS with 0.1% Saponin for 3 minutes/wash. After the washing procedure, the slides were blocked for 15 minutes with 1% normal horse serum in PBS-Saponin, and then were blocked using an Avidin and Biotin kit (Vector, catalog number SP-2001) for 15 minutes each. Thereafter, a mouse monoclonal anti-HMGB1 antibody (2G7 HMGB1 mAb, Critical Therapeutics, Inc., Lexington, Mass.; 0.625 μg/ml), mouse-anti TNP-α antibody (Biosite, San Diego, Calif.; Catalog No. H86410M) and/or mouseanti IL-1β antibody (Immunocontact, Frankfurt, Germany; Catalog Nos. 211-44-531 (1.67 μg/ml) and 211-44-131 (8.33 μg/ml)) were added and incubated with the slides overnight at room temperature in a humid chamber. Mouse IgG2b and IgG1 antibodies (DakoCytomation, Cat. Nos. X0944 (0.625 μg/ml), X0931 (8.33 μg/ml)) of irrelevant specificity were used as controls. The slides were washed three times for 3 minutes/wash in PBS with 0.1% Saponin, then were incubated with biotinylated horse anti-mouse IgG antibody (Vector Laboratories, Burlingame, Calif.; Catalog No. BA-2001; 5 μg/ml), which was diluted in PBS-Saponin containing 1% normal horse serum for 30 minutes. The slides were then treated with peroxidase-conjugated ExtrAvidin (Sigma, St. Louis, Mo.; Catalog No. B-2886) for 45 minutes in the dark and were developed with a DAB kit (Vector Laboratories, Burlingame, Calif.; Catalog No. SK-4100) for 10 minutes. The slides were counterstained with Mayer's hematoxylin and were mounted using a 1:9 dilution of PBS:glycerol. All slides were analyzed under a microscope (Leica Microsystems).

### Evaluation

The stained slides were coded and analyzed independently by two persons who were blind for the purposes of this study. The entire section was analyzed by traditional microscopy evaluation using a Polyvar II microscope (Reichert-Jung, Vienna, Australia). For the evaluation of cytokine expression, the section was divided into different parts: epidermis, dermal infiltrate and dermal non-infiltrate, respectively. The amount of positively stained cells (%) was estimated in each part. To investigate the distribution of HMGB1, nuclear, cytoplasmic and extracellular staining was estimated as a percentage of the total staining in each part of the section. The mean values of the evaluations by the two observers were calculated and used for statistical analyses.

### Statistical Analysis

Statistical analysis was performed using the nonparametric Mann-Whitney U test. p-values less than 0.05 were considered to be significant.

### Results

Increased Expression and Extracellular Deposition of HMGB1 in Active Lesions from Patients with Cutaneous Lupus

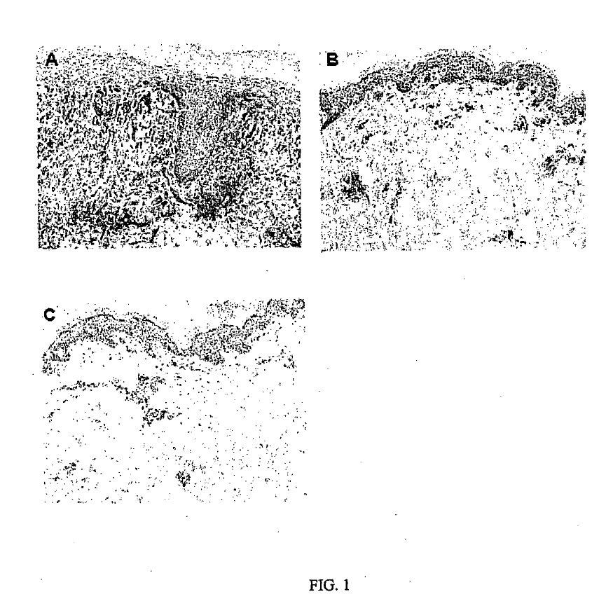

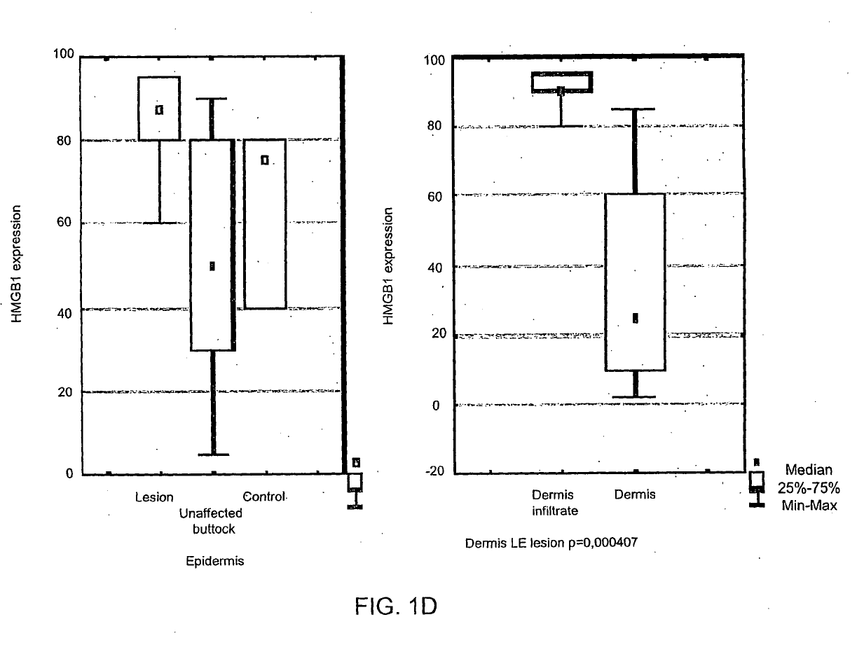

HMGB1 was expressed in both affected and unaffected skin specimens from CLE patients (FIGS. 1A and 1B, respectively), and in skin specimens from healthy control patients (FIG. 1C). The degree of HMGB1 protein expression was consistently higher in both the lesional dermis and epidermis of the affected skin sample, in comparison to the level of HMGB1 protein expression in unaffected buttock skin of the same patient (p<0.001 and p<0.01) (FIG. 1D) and in controls. Infiltrates of mononuclear cells dominated the skin lesions, and within the infiltrates, high levels of HMGB1 expression was observed (FIG. 1A). In the non-infiltrated part of the dermis, the expression of HMGB1 was low and similar to the level of HMGB1 expression in corresponding areas of healthy buttock skin. In both unaffected buttock skin from CLE patients (FIG. 1B) and skin from the control subjects (FIG. 1C), HMGB1 was expressed mainly in the epidermis.

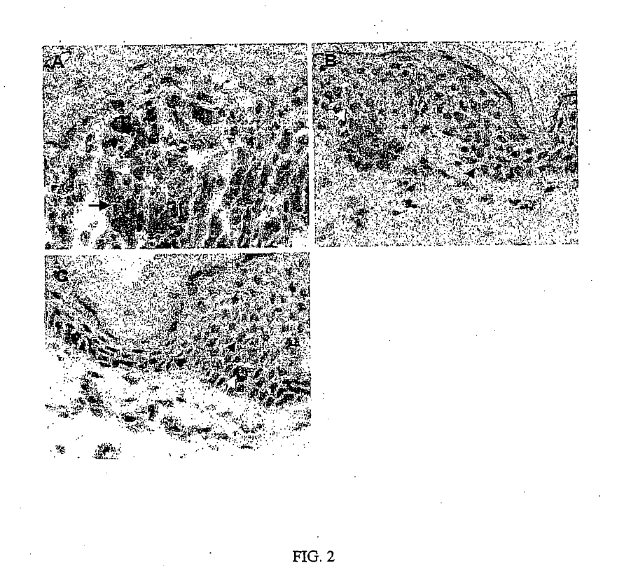

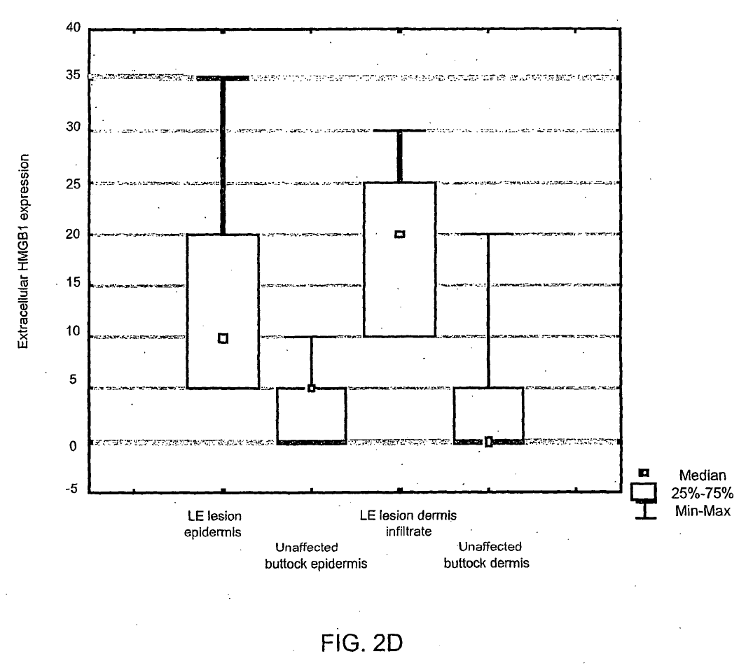

The intracellular localization of HMGB1 was predominately cytoplasmic in the dermis and epidermis of skin biopsies from all patients (FIG. 2). However, translocation of HMGB1 to the extracellular space was detected almost exclusively in the dermis and epidermis of skin biopsies from the lesions of CLE patients (compare FIG. 2A to FIGS. 2B and 2C). The degree of extracellular HMBG1 staining was highly significant for these locations (p<0.001 and p<0.01 for the dermis and epidermis, respectively). In biopsies from healthy control subjects, no extracellular staining for HMGB1 was observed (FIG. 2C).

### TNF-α and IL-1β are Co-Expressed in Areas of Skin Specimens Characterized by Extracellular HMGB1

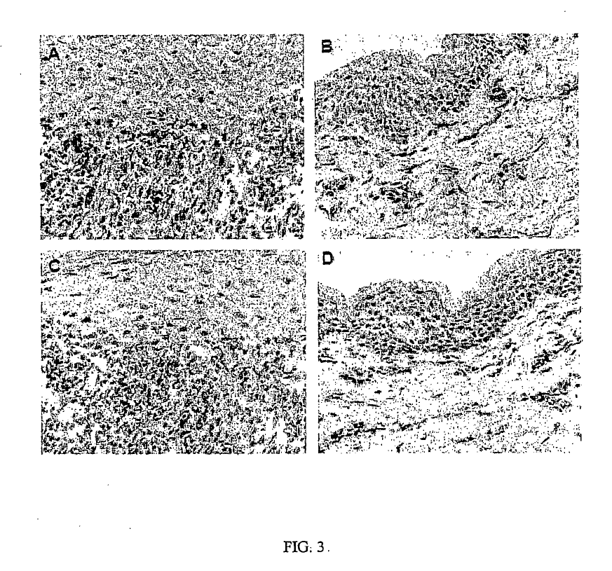

TNF-α expression was detected in the dermis of all subjects, but to a higher degree in the infiltrates of lesions from CLE patients (compare FIG. 3A to FIG. 3B). The localization of TNF-α was mainly intracellular in both the dermis and epidermis of all patients. However, in infiltrates of dermal lesions, extracellular TNT-α was observed to almost the same degree as intracellular TNF-α (FIG. 3A).

IL-1β was expressed in both affected and unaffected skin specimens (FIGS. 3C and 3D), where the most intense staining was found in the epidermis. The level of IL-1β expression was similar in both lesions and unaffected buttock skin (FIGS. 3C and 3D). In both the dermis and epidermis of all patients, the localization of IL-1β was mainly intracellular. Secreted IL-1β was observed only in the dermal infiltrates of lesions (FIGS. 3C and 3D). Control staining with an irrelevant isotype-matched control antibody was negative.

### TNF-α Single Nucleotide Polymorphism (SNP) Analysis

DNA was extracted from peripheral blood mononuclear cells of the CLE patients and was analyzed for the previously-defined −308 TNF single nucleotide polymorphism (SNP) in the TNF-α promoter. Five out of ten patients with CLE had a GG genotype while the carrier frequency of the A allele was 50%. Patients carrying the A allele did not show higher expression of TNF-α in either affected or unaffected skin when compared to the patients with the GG genotype.

### Discussion

Cutaneous lupus erythematosus is the most common form of lupus, and mucocutaneous symptoms constitute 4/11 of the ACR criteria for SLE (Tan, E. M., et al., Arthritis Rheum. 25:1271-1277 (1982)). Although lupus is a heterogenous disease, study of the pathogenesis in skin biopsies is an attractive model as it offers access to directly affected tissue as well as control tissue from the same patient. The appearance of lesions is commonly triggered by UV radiation, which induces the production of TNF-α, and results in a lichenoid tissue reaction pattern with apoptotic cells and a dermal inflammatory infiltrate dominated by T cells. To date, TNF-based approaches for treatment have not been successful for treating lupus. To investigate and identify the role of another factor in lupus, we studied the expression and release of the proinflammatory cytokine, HMGB1, in skin biopsies from patients with SCLE and DLE lesions.

The protein HMBG1 has been shown to play a role in the pathogenesis of particular human inflammatory diseases, including acute and chronic diseases (Andersson, U., et al., J. Leukocyte Biol. 72:10841091 (2002)). Two separate pathways for HMGB1 secretion have been described; either passively from the nuclei of necrotic or damaged cells or actively from activated mononuclear phagocytes (Wang, H., et al., Science 285(5425):248-51 (1999); Scaffidi P., et al., Nature 418(6894):191-195 (2002)). Apoptotic cells fail to release HMGB1 and do not mediate an inflammatory response, even after undergoing secondary necrosis.

As described herein, it was found that both keratinocytes and dermal mononuclear inflammatory cells of skin biopsies from CLE patients exhibited an increased amount of cytoplasmic and extracellular HMGB1, as compared to healthy buttock skin of the same patients. The extracellular HMGB1 staining indicates either release of cytoplasmic HMGB1 from activated macrophages or from necrotic cells, and as necrosis is not typically seen in lupus, it is likely that the extracellular HMGB1 observed in lupus patients is secreted from activated cells. Additionally, keratinocytes could also release HMGB1, which could constitute a novel source contributing to the extracellular pool of HMGB1.

A biallelic polymorphism at position −308 within the human TNF promoter region has been described in SCLE (Werth, V. P., et al., J. Invest. Dermatol. 115:726-730 (2000)) and related to increased TNF-α production. As described herein, none of the patients had the rare AA genotype, although 50% carried the −308A allele. No increased TNF-α expression was observed in the A-allele-carrying patients, as compared to the other patients, although increased TNF-α was observed in all lesions, as compared to unaffected skin. UV radiation causes the release of TNF-α and IL-1 from the keratinocytes (Kock. A., et al., J. Exp. Med. 172(6):1609-1614 (1990); Kupper, T. S., et al., J. Clin. Invest. 80:430-436 (1987)). Both TNF-α and IL-1β can induce secretion of HMGB1, which in turn can stimulate the synthesis of TNF-α and IL-1β. Accordingly, while UV radiation may initiate formation of the lesions, HMGB1 may appear at a later stage, and be of importance in sustaining the inflammation and leading to a more chronic disease.

### Example 2

### CLE-Induced Photosensitivity is Associated with Changes in the Expression of HMGB1 Protein in the Epidermis and Dermis of Affected Individuals

### Introduction

Cutaneous lupus erythematosus (CLE) is a chronic autoimmune skin disease. The majority of patients diagnosed with CLE display photosensitivity, or abnormal sensitivity to sunlight. This condition is characterized by the formation of severe lesions (i.e., CLE lesion flare) that can manifest up to 2 weeks after exposure to sunlight and often last longer than a week. CLE patients have a decreased threshold for induction of erythema after exposure to UV irradiation (UV R) (Orteu, C. H., et al., Photodermatol. Photoimmunol. Photomed. 17(3):95-113 (2001)). Both UVB and UVA irradiation (UVB R and UVA R), and in some cases visible light, can induce lesions in CLE patients (Orteu, C. H., et al., Photodermatol. Photoimmunol. Photomed. 17(3):95-113 (2001); Sanders, C. J., et al., Br. J. Dermatol. 149(1):131-137 (2003)). Notably, the condition of most patients with systemic lupus erythematosus (SLE) becomes exacerbated 3-6 months after summer, when the sun is most intense (Leone, J., et al., Rev. Med. Interne 18(4):286-291 (1997)). Although SLE patients display a high incidence of photosensitivity, the mechanism(s) by which UV R exposure induces CLE lesion flare is unclear. To investigate a potential role for HMGB1 protein in CLE lesion flares, HMGB1 expression levels were analyzed in skin samples from both CLE and healthy patients. Changes in HMBG1 expression in CLE lesions induced by exposure to UVB R also were monitored.

### Materials and Methods

### Subjects

Five CLE patients with documented photosensitivity, and one healthy control patient, participated in the study. All participants gave informed consent. The CLE patients consisted of 4 women and I man, as women display about a 7-fold higher incidence of SCLE photosensitivity than men (Sontheimer, R. D., Lupus 6(2):84-95 (1997)). The average age of the participants was 50 years and the disease had been diagnosed within 3-10 years. The patients did not use any medication during the course of this study. Before commencing photo-provocation (see below), a full clinical investigation was performed for each patient and blood samples were obtained for serological analysis. Positivity for Ro/SSA auto-antibodies was established in 2 of the 5 patients. The study was approved by a local ethical committee.

### Photo-Provocation Protocol

The protocol comprised several steps. First, a minimal erythema dose (ED), defined as barely-perceptible erythema with at least 3 visible corners (Hasan, T., et al., Br. J. Dermatol. 4(4):471-475) was established by irradiating small areas of the patients' middle backs with different doses of UVB rays. Photo-provocation was achieved by administering two or three MEDs of UVB irradiation, with an average dose of 32 mJ/cm² and a mean time of 174 seconds. A 5 cm×8 cm area of the lateral back was exposed to the UVB source. The procedure was repeated 3 times on consecutive days. All patients were monitored and the photo-provoked area was checked initially 24 h after exposure, then every 4-7 days for up to 5 weeks following provocation. The healthy control patient was exposed to UVB using a similar protocol.

### Collection of Skin Biopsies

4 mm punch skin biopsies from the provoked area were taken according to the appearance of clinical symptoms, i.e., at the moment when the lesion erupted and the level of inflammation was highest (e.g., displaying both erythema and infiltration, and, in one case, papules). This usually happened 3-7 days after photo-provocation. The second biopsy was taken, on average, 10 days after irradiation, when clinical symptoms were still intense. The next sampling was performed when the lesions started to dissolve, and only erythema was still apparent. In two patients, redness of the exposed skin area was prolonged and the final biopsies were taken 17 and 27 days after the start of the photo-provocations. Only one biopsy, taken 4 days after irradiation, was obtained from the UVB-exposed skin of the healthy control. A sample from healthy, neon-exposed skin of the buttock was taken from all participants in the study as a control.

### Immunohistochemistry

After a punch biopsy was taken, the tissue was snap frozen in liquid nitrogen, and stored at −70° C. until processed for immunohistochemistry. The biopsies taken at the time point when the most severe clinical features manifested were also sent for histological analysis.

8 μm biopsy sections were obtained using a cryostat, at a chamber temperature of −22° C. and an object temperature of −24° C. The sections were placed on positively-charged gelatin-coated chrome objective glasses, air-dried for 30 min, fixed in 2% formaldehyde in phosphate-buffered saline (PBS), and then frozen at −70° C. until staining. All solutions were at a pH of 7.4. Before staining, the sections were permeabilized in PBS-0.1% Saponin solution for 10 min, and then blocked with hydrogen peroxide solution (1% H₂O₂, 2% NaN₃, 0.1% Saponin in PBS) for 60 min in the dark. The slides were rinsed with 0.1% Saponin in PBS solution three times for 3 min per wash between all procedures. After washing, the slides were blocked for 15 min with 1% normal goat serum in PBS-Saponin, then were blocked with Avidin and Biotin, which were provided by Vector as a kit. Thereafter, the prepared tissues were incubated overnight with rabbit polyclonal anti-HMGB1 antibody (Pharmingen) at concentration of 0.625 μg/ml in a humid chamber. A salivary gland biopsy that was taken from a patient diagnosed with Sjögren syndrome served as a positive control. After approximately 24 h incubation with primary antibodies, the objective glasses were washed with PBS-Saponin solution, and the secondary biotinylated goat anti-rabbit antibodies were diluted with PBS-Saponin and normal goat serum at a concentration of 1.87 μg/ml. The tissues were incubated with the secondary antibody solution in a humid chamber for 45 min in darkness. Afterwards, the slides were washed and stained with DAB solution (diaminobenzadiazine, hydrogen peroxide and buffer) (Vector) for 10 min, and then were washed with PBS-Saponin and finally were washed with PBS. The tissues were dyed in hematoxylin to stain the nuclei of the cells, then were washed in water, dried and mounted using a 1:9 dilution of PBS:glycerol.

### Evaluation of HMGB1 Staininig

The stained slides were coded and analyzed in a blinded, semi-quantitative way. The entire section was analyzed by traditional light microscopy using a Polyvar II microscope (Reichert-Jung, Vienna, Austria). To evaluate HMBG1 expression, the entire skin section was divided into different parts: epidermis, dermal non-infiltrate, and dermal infiltrate, and the total amount of HMGB1 positive cells was established. The percent of cells displaying immunoreactivity (IR) in the cytoplasm and nuclei was determined separately. Extra-cellular staining was assessed by determining what percentage of the stained area was occupied. To account for variations of HMBG1 expression between the patients, the highest HMGB1 expression within one sample was set at 1, while the other numbers were determined as a ratio.

### Histological Assessment

In three of five patients, CLE-specific histological findings were established. A polymorphic light eruption, which is associated with CLE (Hasan, T., et al., Br. J. Dermatol. 136(5):699-705)) was provoked in one of the patients, and a change in HMBG1 expression induced in another patient, were determined not to be CLE-related.

### Statistical Analysis

To assess HMBG1 expression differences between the CLE patients and the healthy control individual, Wilcoxon matched pairs and Mann-Whitney tests were used. More detailed statistical calculations were not available, because only one control subject was used. A p<0.05 value was determined to be statistically-significant. Calculations were performed by STATISTICA 7.0 program (StatSoft Inc, USA).

### Results

HMGB1 expression was detected in healthy and UVB-irradiated skin of both CLE patients and the healthy control individual. In CLE patients, exposure to UV rays changed the pattern of HMGB1 expression, which followed the appearance of clinical symptoms.

### HMGB1 Expression in Epidermis

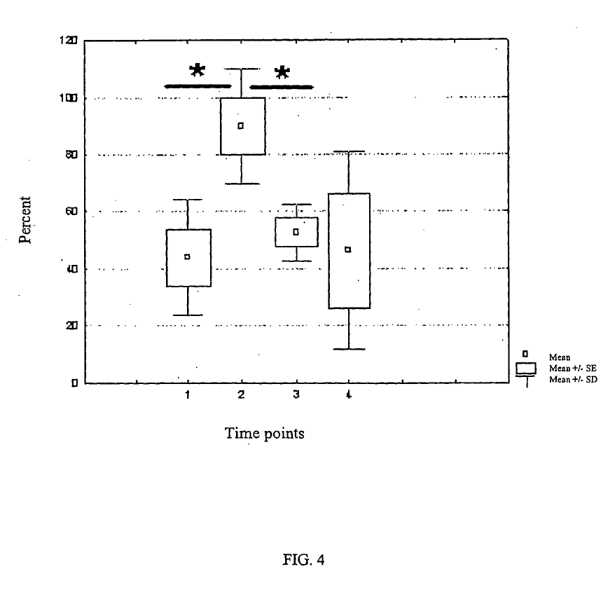

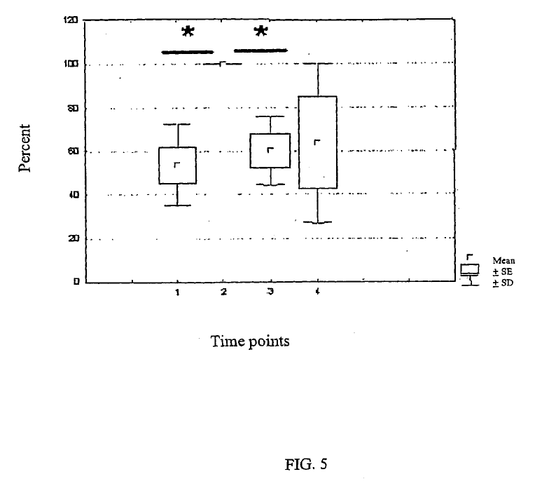

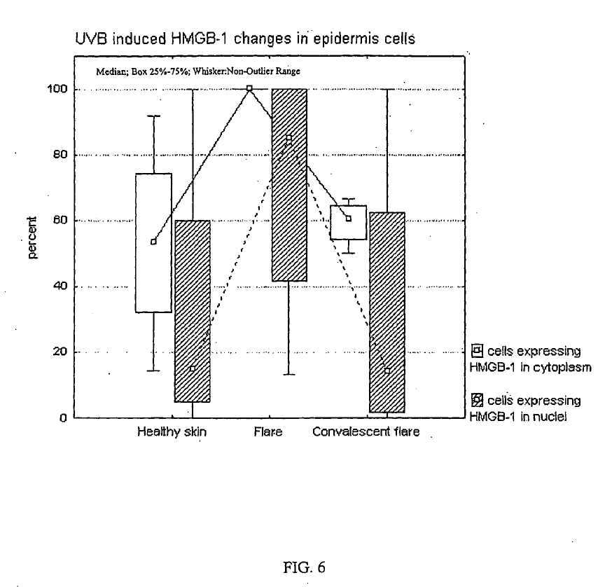

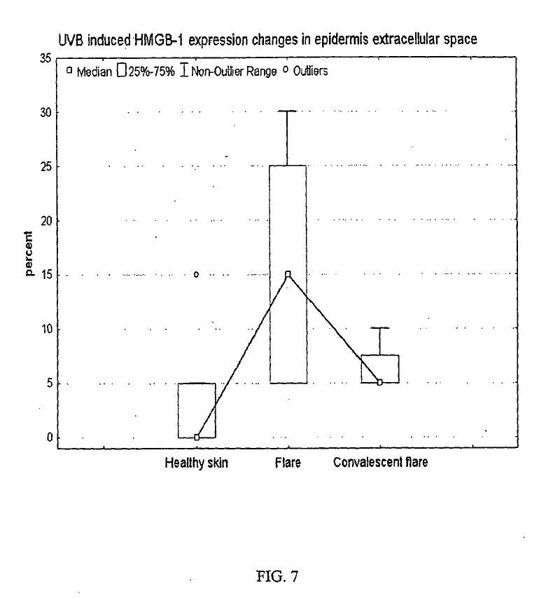

In all CLE patients, the total number of HMBG1-positive epidermal cells increased after UVB-induced flare, in comparison to the patients' healthy buttock skin (FIG. 4). Increased staining was most evident in the cytoplasm of the cells, which increased from 23% to 48% (p<0.05) (FIG. 5). One week after the onset of flare, HMBG1 levels in the cytoplasm of keratinocytes were reduced (p<0.05) to pre-irradiation levels and remained at the same level for a week, while the clinical symptoms dissolved (FIG. 6). Nuclear HMGB1 expression in the cytoplasm of keratinocytes increased from 2% to 5% after provocation (p<0.05). At the moment of flare induction, the amount of extracellular HMGB1 appeared to increase in the epidermis (p=0.055) (FIG. 7).

In the healthy control individual, the maximum amount of HMBG1 staining was documented prior to UVB exposure. After exposure, the total amount of HMGB1 staining decreased, in both the cytoplasm and nuclei, from 100% to 50% and from 100% to 0%, respectively.

### HMGB1 Expression in Dermal Non-Infiltrate

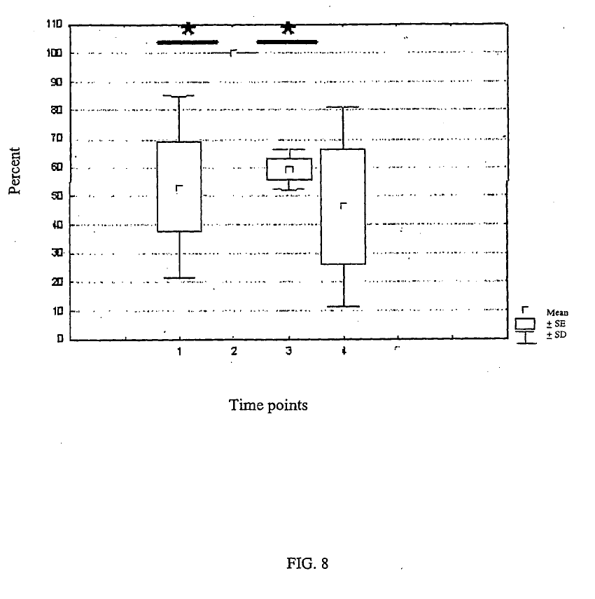

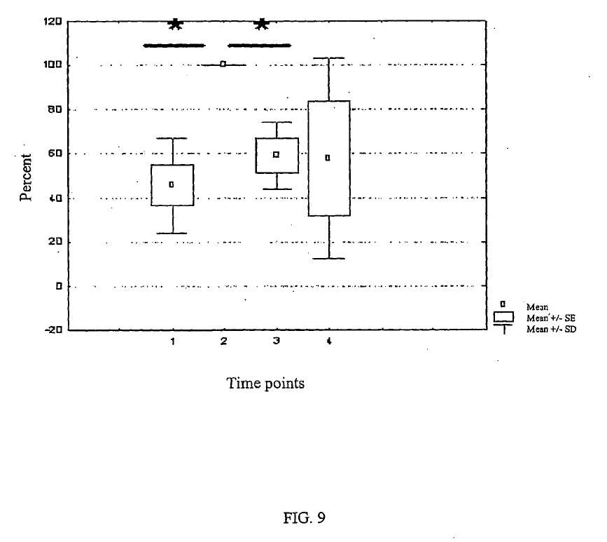

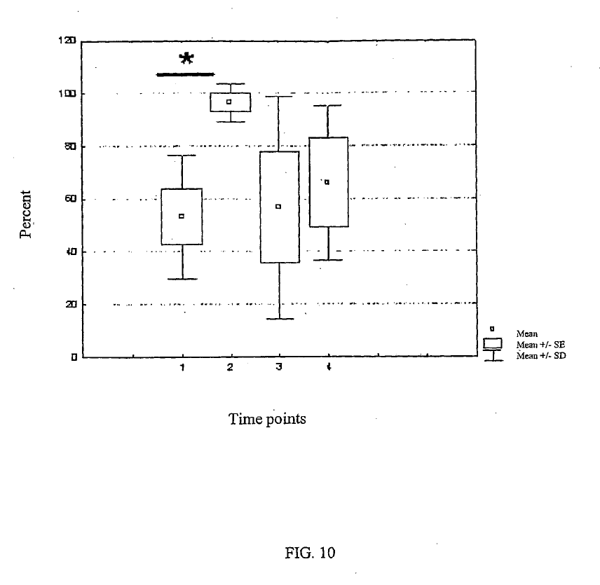

UVB exposure also induced an increase in the amount of HMGB1 positive cells at the onset of lesion flare (FIG. 8), in both the cytoplasm (FIG. 9) and nuclei (FIG. 10) of dermis cells of CLE patients (p<0.05), from 23% to 44% and from 17% to 30%, respectively. Within several days, HMGB1 staining in the cytoplasm was reduced to pre-photo-provocation levels. Late HMGB1 expression in cell nuclei appeared to be decreased. Dermis cells that assembled into groups, though too small to be called infiltrates, tended to have more HMGB1 staining than scattered cells.

In cells from the control individual, exposure to UVB R reduced the level of HMGB1 protein expression in all investigated cellular compartments, for example, from 53% to 40% in cytoplasm, and from 30% to 10% in nuclei.

Significant changes in extracellular HMGB1 levels in non-infiltrated dermis were not detected in CLE patient samples. In cells of the control individual, UVB irradiation increased the abundance of extracellular HMBG1 from 11% to 40%.

### HMGB1 Expression in Dermal Infiltrate

Photo-provocation induced dermal infiltrates in three of five patients, as determined by a high abundance of infiltrating cells and the destruction of a limit between the dermis and epidermis. However, in one of these patients, CLE-specific changes were not confirmed histologically. Mononuclear cells, which dominated the infiltrates, displayed HMGB1 expression levels that were up to 20 fold higher in cytoplasm and up to 2.3 fold higher in nuclei, relative to cells found in non-infiltrated dermis. Furthermore, HMGB1-stained areas were about 1.25-6 times larger in the samples displaying dermal infiltrates, as compared to non-infiltrated dermis. We observed a pattern of changes in HMBG1 expression in response to UVB irradiation that was similar to the patterns observed in cells of the epidermis and non-infiltrated dermis (FIG. 11).

Our results show that UVB exposure upregulated HMGB1 expression in patients diagnosed with CLE. In general, photo-provocation increased HMGB1 abundance in all tissue compartments that were examined in patient samples. In the epidermis of healthy buttock'skin, HMGB1 protein expression was higher in the cytoplasm than in nuclei. UVB irradiation increased HMGB1 expression in both compartments, however, nuclear staining was very low in comparison to cytoplasmic staining. UVB R also increased extracellular HMGB1 levels in the epidermis of CLE patients. Comparatively high cytoplasmic HMBG1 expression was observed in the epidermis of control buttock skin, which may indicate that pathological processes related to CLE are taking place continuously, even in areas where no lesion has formed.

Photo-provocation induced CLE lesions in 3 patients. While comparing infiltrated and non-infiltrated dermis from these patients, we noticed HMGB1 expression in the cytoplasm, nuclei and the extracellular spaces. These results are consistent with HMBG1 functioning as a cytokine, which translocates from the nucleus to the cytoplasm and then to the extracellular spaces.

Fluctuation of HMGB1 expression followed both lesion formation and disappearance, with the highest levels of HMGB1 being observed at the time of flare. Increases in both cellular and extracellular HMGB1 levels suggest that UV rays can facilitate HMBG1 synthesis in both keratinocytes and dermis mononuclear cells and can stimulate HMGB1 release to the extracellular environment, as well.

### Example 3

### Inducible Expression and Secretion of HMGB1 in Human Skin Keratinocytes at Birth

### Introduction

Erythema Toxicum Neonatorum is an acute, innate immune response of transitory duration, that manifests at birth when microbes penetrate into the skin of the human newborn. Histologically, the rash (FIG. 12) is characterized by an upregulation of proinflammatory activity and a local recruitment of immunocytes, including macrophages. High mobility group box chromosomal protein 1 (HMBG1) is a proinflammatory cytokine that is released by macrophages in response to microbial challenge. Here, we reasoned that keratinocytes might secrete HMGB1 in response to the first colonization of the skin by microbes in human newborns and that HMBG1-mediated inflammation might play a role in the development and progression of Erythema Toxicum and other inflammatory skin conditions.

### Materials and Methods

### Patient Samples

Punch biopsies of 3 mm were obtained after local anaesthesia from 6 infants with, and 4 infants without, Erythema Toxicum, and from four healthy adults, as previously described (Marchini G., et al., Ped. Dermatol 198-177-87 (2001)). All infants were healthy and had an uncomplicated delivery and neonatal period. Furthermore, all were born at term and were exclusively breastfed. The ethics committee of the Karolinska Hospital approved this study and all parents provided informed consent.

### Imunohistochemistry

Biopsies were fixed for 75 minutes in Lanasfix (Bie & Berntsen, Denmark), containing 4% paraformaldehyde and 14% picric acid in 0.1 M phosphate buffer, and were thereafter rinsed for at least 24 hours in phosphate buffer containing 10% sucrose. The biopsies were frozen and embedded in isopentan and OCT-compound (Sakura, Netherlands), and 9-10 μm sections were prepared. Endogenous peroxidase activity was blocked with hydrogen peroxide in phosphate-buffered saline (PBS), supplemented with 2% sodium azide and 0.1% saponin. All subsequent steps were carried out in PBS/saponin buffer. The slides were incubated overnight at room temperature in a humid chamber with either rabbit polyclonal HMBG1 antibodies (BD Biosciences Pharmingen, San Jose, Calif.) or mouse monoclonal HMGB1 antibodies (Critical Therapeutics, Inc, Cambridge, Mass.), at a concentration of 0.125 μg/ml and 0.038 μg/ml, respectively. Control staining was performed in parallel experiments by excluding the primary antibody for the procedure using rabbit polyclonal HMGB1 antibodies and using species and isotype-matched antibodies (i.e., mouse IgG1 antibody (DAKO, Glostrup, Denmark) for the procedure with the mouse monoclonal HMGB1 antibodies. The slides were washed and incubated with normal goat (for the procedure using the rabbit polyclonal anti-HMGB1 antibodies) or horse serum (for the procedure using the mouse monoclonal HMGB1 antibodies), respectively, followed by incubation with the biotinylated secondary antibodies, goat anti-rabbit IgG diluted in 1% normal goat serum, and horse anti-mouse IgG1 in normal horse serum. Subsequently, the slides were incubated with an avidin-biotin-horseradish peroxidase complex (ABC-elite, Vector Laboratories, Burlingame, Calif.) and the brown color reaction was developed using 0.5 mg/ml diaminobenzidine (DAB-kit, Vector Laboratories, Burlingame, Calif.). Counterstaining was performed in Mayer's 10% haematoxylin (Merck, Darmstadt, Germany) and the slides were mounted in Kaiser's glycerin-gelatin (Merck, Darmstadt, Germany). Two tissue sections were examined for each type of HMBG1 antibody. An estimation of the number of cells with cytoplasmic HMBG1 staining for both the monoclonal and polyclonal antibody, respectively, was made using a semi-quantitative scale ranging from 0 to ++++, wherein 0=no positive cell; +=<25%, ++=25-50%; +++=50-75%; and ++++=>75%. All sections were analyzed using an Axioplan Universal microscope (Zeiss, Jena, Germany).

Double Immunofluorescent Staining of HMGB1 with Mac387, LAMP1; LAMP2 and EEA1

Cryosectioned samples, fixed as described above, were blocked with 4% normal goat serum in PBS with 1% bovine serum albumin (BSA) and 0.1% saponin by incubation for 1 hour at room temperature. Sections were incubated in blocking buffer overnight at 4° C. with the following primary antibodies anti-Mac 387 monoclonal mouse anti-human myeloid/histocyte antigen (Dako, Glostrup, Denmark); anti-LAMP1 and anti-LAMP2 mouse IgG1 monoclonal antibodies (DSHB, Iowa city, Iowa); and anti-EEA1 mouse IgG1 monoclonal antibody (BD, Bioscience, Milan, Italy). After rinsing, the slides were incubated for 1 hour at room temperature with the following secondary fluorescent antibodies: goat anti-mouse Alexa 488(Molecular Probes, Eugene, Oreg.) for the cell surface markers Mac387, LAMP1, LAMP2 (IgG1, DSHB, Iowa City, Iowa) and Early Endosomal Antigen1 (EEA1) (IgG1; BD Bioscience, Milan, Italy), and goat anti-rabbit Alexa 546 (Molecular Probes, Eugene, Oreg.) for HMGB1 (Y3D Biosciences Pharmingen, San Jose, Calif.). The slides were washed with 1% BSA and 0.1% saponin in PBS, and nuclei were counterstained with 4′,6-Diamidino-2-phenylindole (DAPI) (Molecular Probes, Eugene, Oreg.). The slides were mounted in Vectashield Hard-set medium (Vector Laboratories, Burlingame, Calif.). Confocal images were captured with a Zeiss LSM 510 META confocal microscope, using a 40×/1.3 NA objective. DAPI was excited at 405 nm and detected at 420-480 nm, Alexa 488 was excited. at 488 nm and detected at 505-530 nm, and Alexa 546 was excited at 543 nm and detected at 560-650,nm. Negative controls for autofluorescence and non-specific binding of secondary antibodies were performed by excluding the primary antibodies from the staining protocol.

### Results

### Immunohistochemistry

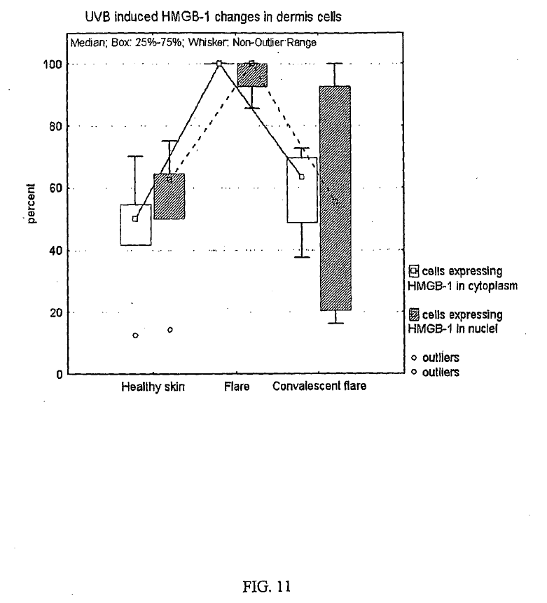

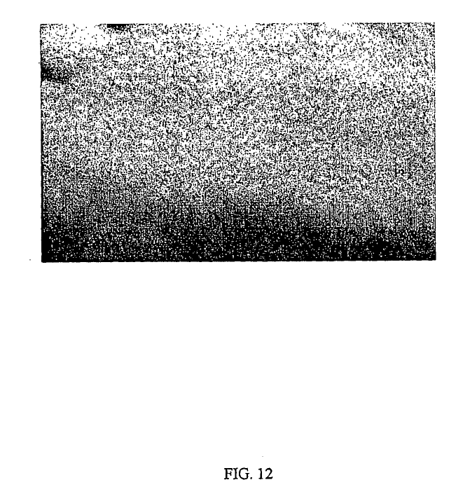

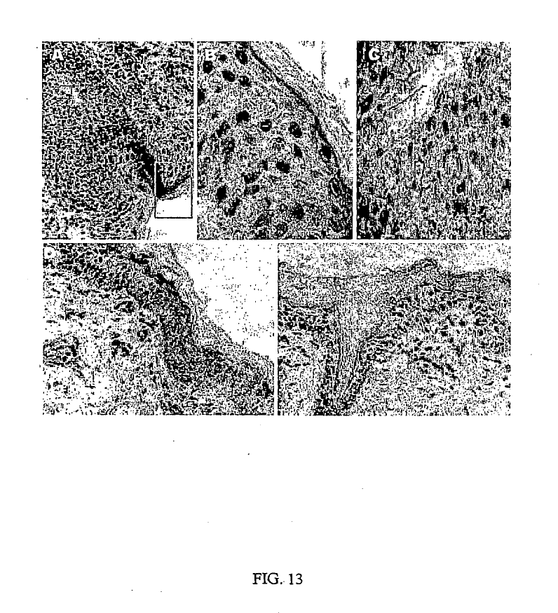

We did not observe any difference between the staining patterns produced by the monoclonal and polyclonal HMBG1 antibodies (see Table 1). In the keratinocytes overriding the hair follicle, and the dermal infiltrate of the lesions of Erythema Toxicum, HMBG1 staining was clearly evident in the cytoplasm and extracellular space (FIGS. 13A, 13B and 13D). Similar results were seen in inflammatory cells that accumulated near the hair follicle (FIG. 13C). Non-inflamed skin from healthy control infants (FIG. 13E), and adults, displayed strong HMBG1 staining that was mainly restricted to the nucleus. HMGB1 was mostly absent from the cytoplasm in these samples.

### Immunofluorescence Analysis and Confocal Microscopy

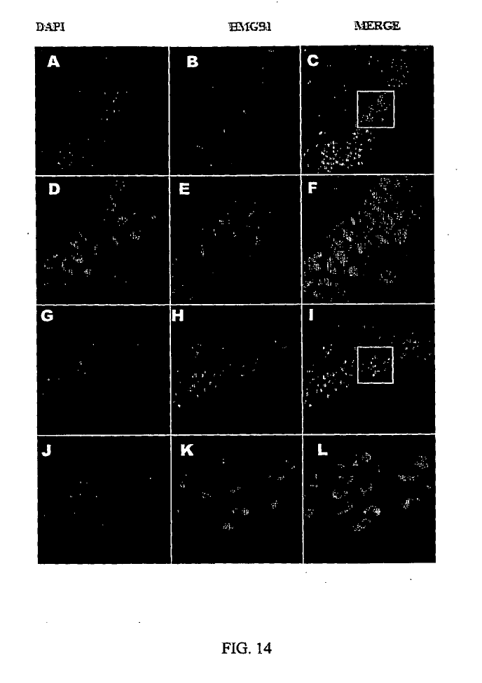

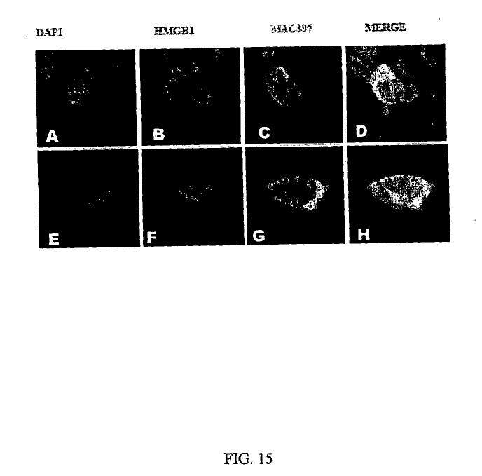

In the keratinocytes of inflammatory lesions, HMBG1 clearly localized to the cytoplasm and extracellular space, as evidenced by the lack of HMBG1 signal in nuclei that had been counterstained with DAPI (FIGS. 14A-F). However, in non inflamed hair-follicles, HMGB1 was restricted to the nucleus (FIGS. 14G-L). Inflammatory cells that were recruited to the dermal infiltrate near the hair follicle also expressed HMGB1 in the cytoplasm and extracellular spaces (FIG. 13C). Double immunostaining and confocal microscopy showed that these cells were Mac387+ macrophages (FIGS. 15A-D). Consistent with HMBG1 localization in keratinocytes near inflammatory lesions, HMGB1 localized to the cytoplasm and extracellular space surrounding Mac387+ macrophages in the dermal infiltrate (FIGS. 15A-D), but was mainly nuclear in Mac387+ macrophages from unaffected skin (FIGS. 15E-H).

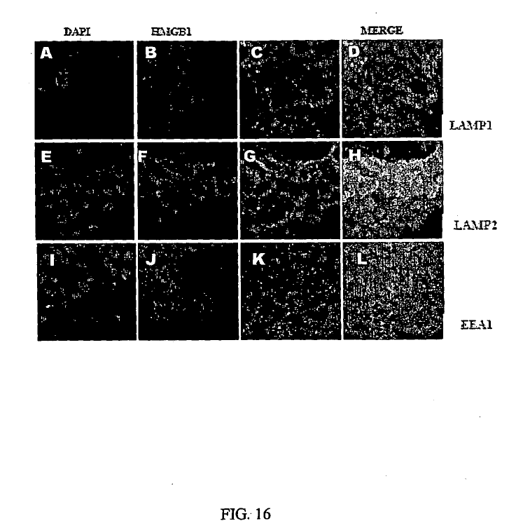

To determine whether secretion of HMBG1 was linked to lysosomal structures, double immunostaining and confocal microscopy was performed using HMGB1 antibodies and antibodies to various lysosomal markers. Co-localization of HMGB1 with the lysosomal markers LAMP1, LAMP2, and EEA1 was not observed (FIG. 16), suggesting that HMGB1 is not secreted and/or stored in conventional lysosomes or early endosomes in skin keratinocytes. Staining for LAMP1, LAMP2, and EEA1, revealed distinct cytoplasmic vesicular structures by immunofluorescence, while HMGB1 staining was more diff-use and uniform (FIG. 16). The images shown in FIG. 16 are from the same biopsy regions as those shown in FIGS. 15A-F.

Here we show, for the first time, that keratinocytes are able to secrete HMBG1. Keratinocytes are active producers of proinflammatory cytokines and chemokines, and thus play a pivotal role in host defense (Wang H., et al. Surgery 126:389-392 (1999)). The secretion of HMGB1 by skin keratinocytes may promote resistance to invading microbes in new-born infants.

The relevant teachings of all publications cited herein not previously incorporated by reference, are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

TABLE 1

| | | | | | | Control- | | | | | | |
|---|---|---|---|---|---|---|---|---|---|---|---|---|
| Erythema Toxicum-Infants | | | | | | Infants | | | Adults | | | |
| 1 | 2 | 3 | 4 | 5 | 6 | 1 | 2 | 3 | 1 | 2 | 3 | 4 |

HMGB1 (mouse-monoclonal)

| | | | | | | | | | | | | | |
|---|---|---|---|---|---|---|---|---|---|---|---|---|---|
| Epidermis | | | | | | | | | | | | | |
| Cytoplasma | +++ | +++ | +++ | +++ | +++ | ++++ | + | + | + | + | + | + | + |
| of keratinocytes | | | | | | | | | | | | | |
| Dermis | | | | | | | | | | | | | |
| Cytoplasma | +++ | +++ | +++ | +++ | +++ | ++ | − | − | − | − | − | − | − |
| of inflammatory cells | | | | | | | | | | | | | |

HMGB1 (rabbit-polyclonal)

| | | | | | | | | | | | | | |
|---|---|---|---|---|---|---|---|---|---|---|---|---|---|
| Epidermis | | | | | | | | | | | | | |
| Cytplasma | +++ | +++ | +++ | +++ | +++ | ++++ | + | + | + | + | + | + | + |
| of keratinocytes | | | | | | | | | | | | | |
| Dermis | | | | | | | | | | | | | |
| Cytoplasma | +++ | +++ | +++ | +++ | +++ | ++ | − | − | − | − | − | − | − |
| of inflammatory cells | | | | | | | | | | | | | |

Staining with monoclonal and polyclonal anti-HMGB1 antibodies, respectively, yielded consistent staining patterns in specimens from infants with the Erythema Toxicum, as well as in specimens from control infants and healthy adults

1. A method of treating an inflammatory skin condition in a subject comprising administering to said subject an HMGB antagonist.

2. The method of claim 1, wherein said inflammatory skin condition is selected from the group consisting of psoriasis, acne, pruritis, rosacea, dermatitis, erythematosus multiforme, erythema toxicum, folliculitis, impetigo, lupus erythematosus (LE), cold sores, dry skin, allergic skin conditions, burns, sunburn, bacterially-mediated inflammatory skin conditions and insect bites.

3-4. (canceled)

5. The method of claim 2, wherein said dermatitis is selected from the group consisting of atopic dermatitis, contact dermatitis, seborrheic dermatitis, nummular dermatitis, exfoliative dermatitis, periorial dermatitis and stasis dermatitis.

6. (canceled)

7. The method of claim 1 further comprising administering one or more additional agents selected from the group consisting of an age spot-removing agent, a keratoses-removing agent, an analgesic, an anesthetic, an antiacne agent, an antibacterial agent, an antiyeast agent, an antifungal agent, an antiviral agent, an antiburn agent, an antidandruff agent, an antidermatitis agent, an antipruritic agent, an antiperspirant, an antiinflammatory agent, an antihyperkeratolytic agent, an antidryskin agent, an antipsoriatic agent, an antiseborrheic agent, an astringent, a softener, an emollient agent, coal tar, a bath oil, sulfur, a rinse conditioner, a foot care agent, a hair growth agent, a powder, a shampoo, a skin bleach, a skin protectant, a soap, a cleanser, an antiaging agent, a sunscreen agent, a wart remover, a vitamin, a tanning agent, a topical antihistamine, a hormone, a vasodilator and a retinoid.

8. The method of claim 1, wherein said HMGB antagonist is selected from the group consisting of a high mobility group (HMGB) A box or a biologically active fragment thereof, an antibody to HMGB or an antigen-binding fragment thereof, an HMGB small molecule antagonist, an antibody to TLR2 or an antigen-binding fragment thereof, a soluble TLR2 polypeptide, an antibody to RAGE or an antigen-binding fragment thereof, a soluble RAGE polypeptide and a RAGE small molecule antagonist.

9-10. (canceled)

11. The method of claim 8, wherein said mammalian HMGB A box or biologically active fragment thereof is a HMBG1 A box or biologically active fragment thereof.

12. The method of claim 11, wherein said HMGB A box or biologically active fragment thereof comprises SEQ ID NO:4.

13. (canceled)

14. The method of claim 8, wherein said HMGB antagonist is an antibody or antigen-binding fragment thereof that binds an HMGB polypeptide or a fragment thereof.

15. (canceled)

16. The method of claim 14, wherein said HMGB polypeptide or fragment thereof is an HMBG1 polypeptide or fragment thereof.

17. The method of claim 16, wherein said HMBG1 polypeptide or fragment thereof consists of SEQ ID NO: 1.

18. The method of claim 14, wherein said HMGB polypeptide or fragment thereof is an HMGB B box or biologically active fragment thereof.

19. The method of claim 18, wherein said HMGB B box or biologically active fragment thereof consists of SEQ ID NO:5 or SEQ ID NO:45.

20-22. (canceled)

23. The method of claim 14, wherein said antibody or antigen-binding fragment thereof is selected from the group consisting of a monoclonal antibody, a chimeric antibody a humanized antibody a human antibody and an antigen-binding fragment of any of the foregoing.

24-26. (canceled)

27. The method of claim 8, wherein said HMGB antagonist is an HMGB small molecule antagonist.

28. The method of claim 27, wherein said HMGB small molecule antagonist is an ester of an alpha-ketoalkanoic acid.

29. The method of claim 28, wherein said ester of an alpha-ketoalkanoic acid is selected from the group consisting of an ester of a C3 to C8 straight chain or branched alpha-ketoalkanoic acid, an ester of pyruvic acid, an ethyl ester, a propyl ester, a butyl ester, a carboxymethyl ester, an acetoxymethyl ester, a carbethoxymethyl ester, an ethoxymethyl ester and ethyl pyruvate.

30-33. (canceled)

34. The method of claim 2, wherein said bacterially-mediated inflammatory skin condition is selected from the group consisting of acne, rosacea, cellulitis, acute lymphangitis, lymphadenitis, erysipelas, cutaneous abcesses, necrotizing subcutaneous infections, staphylococcal scalded skin syndrome, folliculitis, furuncles, hidradenitis suppurativa, carbuncles, paronychial infections and erythasma, nummular dermatitis and perioral dermatitis.

35-41. (canceled)

42. A method of inhibiting release of HMBG1 from keratinocytes comprising administering an HMGB antagonist.

43. A method of treating melanoma comprising administering to a subject an HMGB antagonist.

44. (canceled)

45. The method of claim 2, wherein said lupus erythematosus (LE) is selected from the group consisting of acute cutaneous lupus erythematosus (ACLE), subacute cutaneous erythematosus (SCLE), chronic cutaneous lupus erythematosus (CCLE), discoid lupus erythematosus (DLE), systemic lupus erythematosus, drug-induced lupus erythematosus and neonatal lupus erythematosus.

46. A method of preventing or decreasing tissue damage from exposure to UV comprising administering an HMGB antagonist.


SEQUENCE LISTING (45 sequences)

SEQ ID NO: 1 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 2 - protein, 215 aa, Mus musculus
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEDDEEDEEDEEEEEEEEDEDEEEDDDDE

SEQ ID NO: 3 - protein, 209 aa, Homo sapiens
MGKGDPNKPRGKMSSYAFFVQTCREEHKKKHPDSSVNFAEFSKKCSERWKTMSAKEKSKFEDMAKSDKARYDREMKNYVPPKGDKKGKKKDPNAPKRPPSAFFLFCSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 4 - protein, 54 aa, Homo sapiens
PDASVNFSEFSKKCSERWKTMSAKEKGKFEDMAKADKARYEREMKTYIPPKGET

SEQ ID NO: 5 - protein, 69 aa, Homo sapiens
NAPKRPPSAFFLFCSEYRPKIKGEHPGLSIGDVAKKLGEMWNNTAADDKQPYEKKAAKLKEKYEKDIAA

SEQ ID NO: 6 - protein, 216 aa, Homo sapiens [sequence omitted]

SEQ ID NO: 7 - protein, 77 aa, Homo sapiens
PTGKMSSYAFFVQTCREEHKKKHPDASVNFSEFSKKCSERWKTMSAKEKGKFEDMAKADKARYEREMKTYIPPKGET

SEQ ID NO: 8 - protein, 74 aa, Homo sapiens
FKDPNAPKRLPSAFFLFCSEYRPKIKGEHPGLSIGDVAKKLGEMWNNTAADDKQPYEKKAAKLKEKYEKDIAAY

SEQ ID NO: 9 - DNA, 648 bp, Homo sapiens
atgggcaaaggagatcctaagaagccgacaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcttccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatatagctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagaagatgaagaagaagatgatgatgatgaa

SEQ ID NO: 10 - protein, 216 aa, Homo sapiens [sequence omitted]

SEQ ID NO: 11 - DNA, 633 bp, Homo sapiens
atgggcaaaggagatcctaagaagccgagaggcaaaatgtcatcatatgcattttttgtgcaaacttgtcgggaggagcataagaagaagcactcagatgcttcagtcaacttctcagagttttctaacaagtgctcagagaggtggaagaccatgtctgctaaagagaaaggaaaatttgaggatatggcaaaggcggacaagacccattatgaaagacaaatgaaaacctatatccctcccaaaggggagacaaaaaagaagttcaaggatcccaatgcacccaagaggcctccttcggccttcttcctgttctgctctgagtatcacccaaaaatcaaaggagaacatcctggcctgtccattggtgatgttgcgaagaaactgggagagatgtggaataacactgctgcagatgacaagcagcctggtgaaaagaaggctgcgaagctgaaggaaaaatacgaaaaggatattgctgcatatcaagctaaaggaaagcctgaggcagcaaaaaagggagttgtcaaagctgaaaaaagcaagaaaaagaaggaagaggaggaagatgaggaagatgaagaggatgaggaggaggaagatgaagaagatgaagaagatgatgatgatgaa

SEQ ID NO: 12 - protein, 211 aa, Homo sapiens
MGKGDPKKPRGKMSSYAFFVQTCREEHKKKHSDASVNFSEFSNKCSERWKTMSAKEKGKFEDMAKADKTHYERQMKTYIPPKGETKKKFKDPNAPKRPPSAFFLFCSEYHPKIKGEHPGLSIGDVAKKLGEMWNNTAADDKQPGEKKAAKLKEKYEKDIAAYQAKGKPEAAKKGVVKAEKSKKKKEEEEDEEDEEDEEEEDEEDEEDDDDE

SEQ ID NO: 13 - DNA, 564 bp, Homo sapiens
atgggcaaaggagaccctaagaagccgagaggcaaaatgtcatcatatgcattttttgtgcaaacttgtcgggaggagtgtaagaagaagcacccagatgcttcagtcaacttctcagagttttctaagaagtgctcagagaggtggaaggccatgtctgctaaagataaaggaaaatttgaagatatggcaaaggtggacaaagaccgttatgaaagagaaatgaaaacctatatccctcctaaaggggagacaaaaaagaagttcgaggattccaatgcacccaagaggcctccttcggcctttttgctgttctgctctgagtattgcccaaaaatcaaaggagagcatcctggcctgcctattagcgatgttgcaaagaaactggtagagatgtggaataacacttttgcagatgacaagcagctttgtgaaaagaaggctgcaaagctgaaggaaaaatacaaaaaggatacagctacatatcgagctaaaggaaagcctgatgcagcaaaaaagggagttgtcaaggctgaaaaaagcaagaaaaagaaggaagaggag

SEQ ID NO: 14 - protein, 188 aa, Homo sapiens
MGKGDPKKPRGKMSSYAFFVQTCREECKKKHPDASVNFSEFSKKCSERWKAMSAKDKGKFEDMAKVDKDRYEREMKTYIPPKGETKKKFEDSNAPKRPPSAFLLFCSEYCPKIKGEHPGLPISDVAKKLVEMWNNTFADDKQLCEKKAAKLKEKYKKDTATYRAKGKPDAAKKGVVKAEKSKKKKEEE

SEQ ID NO: 15 - DNA, 615 bp, Homo sapiens
atggacaaagcagatcctaagaagctgagaggtgaaatgttatcatatgcattttttgtgcaaacttgtcaggaggagcataagaagaagaacccagatgcttcagtcaagttctcagagtttttaaagaagtgctcagagacatggaagaccatttttgctaaagagaaaggaaaatttgaagatatggcaaaggcggacaaggcccattatgaaagagaaatgaaaacctatatccctcctaaaggggagaaaaaaaagaagttcaaggatcccaatgcacccaagaggcctcctttggcctttttcctgttctgctctgagtatcgcccaaaaatcaaaggagaacatcctggcctgtccattgatgatgttgtgaagaaactggcagggatgtggaataacaccgctgcagctgacaagcagttttatgaaaagaaggctgcaaagctgaaggaaaaatacaaaaaggatattgctgcatatcgagctaaaggaaagcctaattcagcaaaaaagagagttgtcaaggctgaaaaaagcaagaaaaagaaggaagaggaagaagatgaagaggatgaacaagaggaggaaaatgaagaagatgatgataaa

SEQ ID NO: 16 - protein, 205 aa, Homo sapiens [sequence omitted]

SEQ ID NO: 17 - DNA, 240 bp, Homo sapiens
atgggcaaaggagatcctaagaagccgagaggcaaaatgtcatcatgtgcattttttgtgcaaacttgttgggaggagcataagaagcagtacccagatgcttcaatcaacttctcagagttttctcagaagtgcccagagacgtggaagaccacgattgctaaagagaaaggaaaatttgaagatatgccaaaggcagacaaggcccattatgaaagagaaatgaaaacctatataccc

SEQ ID NO: 18 - protein, 80 aa, Homo sapiens
MGKGDPKKPRGKMSSCAFFVQTCWEEHKKQYPDASINFSEFSQKCPETWKTTIAKEKGKFEDMPKADKAHYEREMKTYIP

SEQ ID NO: 19 - DNA, 240 bp, Homo sapiens
aaacagagaggcaaaatgccatcgtatgtattttgtgtgcaaacttgtccggaggagcgtaagaagaaacacccagatgcttcagtcaacttctcagagttttctaagaagtgcttagtgagggggaagaccatgtctgctaaagagaaaggacaatttgaagctatggcaagggcagacaaggcccgttacgaaagagaaatgaaaacatatatccctcctaaaggggagacaaaaaaa

SEQ ID NO: 20 - protein, 80 aa, Homo sapiens
KQRGKMPSYVFCVQTCPEERKKKHPDASVNFSEFSKKCLVRGKTMSAKEKGQFEAMARADKARYEREMKTYIPPKGETKK

SEQ ID NO: 21 - DNA, 258 bp, Homo sapiens
atgggcaaaagagaccctaagcagccaagaggcaaaatgtcatcatatgcattttttgtgcaaactgctcaggaggagcacaagaagaaacaactagatgcttcagtcagtttctcagagttttctaagaactgctcagagaggtggaagaccatgtctgttaaagagaaaggaaaatttgaagacatggcaaaggcagacaaggcctgttatgaaagagaaatgaaaatatatccctacttaaaggggagacaaaaa

SEQ ID NO: 22 - protein, 86 aa, Homo sapiens
MGKRDPKQPRGKMSSYAFFVQTAQEEHKKKQLDASVSFSEFSKNCSERWKTMSVKEKGKFEDMAKADKACYEREMKIYPYLKGRQK

SEQ ID NO: 23 - DNA, 211 bp, Homo sapiens
atgggcaaaggagaccctaagaagccaagagagaaaatgccatcatatgcattttttgtgcaaacttgtagggaggcacataagaacaaacatccagatgcttcagtcaactcctcagagttttctaagaagtgctcagagaggtggaagaccatgcctactaaacagaaaggaaaattcgaagatatggcaaaggcagacagggcccata

SEQ ID NO: 24 - protein, 70 aa, Homo sapiens
MGKGDPKKPREKMPSYAFFVQTCREAHKNKHPDASVNSSEFSKKCSERWKTMPTKQKGKFEDMAKADRAH

SEQ ID NO: 25 - protein, 54 aa, Homo sapiens; identical to SEQ ID NO: 4

SEQ ID NO: 26 - protein, 53 aa, Homo sapiens
DSSVNFAEFSKKCSERWKTMSAKEKSKFEDMAKSDKARYDREMKNYVPPKGDK

SEQ ID NO: 27 - protein, 54 aa, Homo sapiens
PEVPVNFAEFSKKCSERWKTVSGKEKSKFDEMAKADKVRYDREMKDYGPAKGGK

SEQ ID NO: 28 - protein, 54 aa, Homo sapiens; identical to SEQ ID NO: 4

SEQ ID NO: 29 - protein, 54 aa, Homo sapiens
SDASVNFSEFSNKCSERWKTMSAKEKGKFEDMAKADKTHYERQMKTYIPPKGET

SEQ ID NO: 30 - protein, 54 aa, Homo sapiens
PDASVNFSEFSKKCSERWKAMSAKDKGKFEDMAKVDKADYEREMKTYIPPKGET

SEQ ID NO: 31 - protein, 54 aa, Homo sapiens
PDASVKFSEFLKKCSETWKTIFAKEKGKFEDMAKADKAHYEREMKTYIPPKGEK

SEQ ID NO: 32 - protein, 54 aa, Homo sapiens
PDASINFSEFSQKCPETWKTTIAKEKGKFEDMAKADKAHYEREMKTYIPPKGET

SEQ ID NO: 33 - protein, 38 aa, Homo sapiens
PDASVNSSEFSKKCSERWKTMPTKQGKFEDMAKADRAH

SEQ ID NO: 34 - protein, 54 aa, Homo sapiens
PDASVNFSEFSKKCLVRGKTMSAKEKGQFEAMARADKARYEREMKTYIPPKGET

SEQ ID NO: 35 - protein, 54 aa, Homo sapiens
LDASVSFSEFSNKCSERWKTMSVKEKGKFEDMAKADKACYEREMKIYPYLKGRQ

SEQ ID NO: 36 - protein, 84 aa, Homo sapiens
GKGDPKKPRGKMSSYAFFVQTCREEHKKKHPDASVNFSEFSKKCSERWKTMSAKEKGKFEDMAKADKARYEREMKTYIPPKGET

SEQ ID NO: 37 - protein, 74 aa, Homo sapiens
FKDPNAPKRPPSAFFLFCSEYRPKIKGEHPGLSIGDVAKKLGEMWNNTAADDKQPYEKKAAKLKEKYEKDIAAY

SEQ ID NO: 38 - protein, 74 aa, Homo sapiens
KKDPNAPKRPPSAFFLFCSEHRPKIKSEHPGLSIGDTAKKLGEMWSEQSAKDKQPYEQKAAKLKEKYEKDIAAY

SEQ ID NO: 39 - protein, 74 aa, Homo sapiens; identical to SEQ ID NO: 8

SEQ ID NO: 40 - protein, 74 aa, Homo sapiens
FKDPNAPKRPPSAFFLFCSEYHPKIKGEHPGLSIGDVAKKLGEMWNNTAADDKQPGEKKAAKLKEKYEKDIAAY

SEQ ID NO: 41 - protein, 74 aa, Homo sapiens
FKDSNAPKRPPSAFLLFCSEYCPKIKGEHPGLPISDVAKKLVEMWNNTFADDKQLCEKKAAKLKEKYKKDTATY

SEQ ID NO: 42 - protein, 74 aa, Homo sapiens
FKDPNAPKRPPSAFFLFCSEYRPKIKGEHPGLSIGDVVKKLAGMWNNTAAADKQFYEKKAAKLKEKYKKDIAAY

SEQ ID NO: 43 - protein, 92 aa, Homo sapiens
FKDPNAPKRPPSAFFLFCSEYRPKIKGEHPGLSIGDVAKKLGEMWNNTAADDKQPYEKKAAKLKEKYEKDIAAYRAKGKPDAAKKGVVKAEK

SEQ ID NO: 44 - protein, 20 aa, Homo sapiens
NAPKRPPSAFFLFCSEYRPK

SEQ ID NO: 45 - protein, 20 aa, Homo sapiens
FKDPNAPKRLPSAFFLFCSE